(19) United States
(12) Patent Application Publication
(10) Pub. No.: US 2026/0361137 A1
(43) Pub. Date: Nov. 12, 2026

(54) WAVESHAPING FOR WIRELESS POWER TRANSFER

(71) Applicant: Whoop, Inc., Boston, MA (US)

(72) Inventor: Elizabeth Anne Sorenti, Medford, MA (US)

(21) Appl. No.: 19/669,219

(22) Filed: May 6, 2026

Related U.S. Application Data
(60) Provisional application No. 63/801,590, filed on May 7, 2025.

(51) Int. Cl. H02J 50/20; A61B 5/024; H02J 7/02; H02J 50/00

(52) U.S. Cl. CPC H02J 50/20; H02J 7/02; A61B 5/02438; H02J 50/001; H02J 2207/20

(57) ABSTRACT

A wireless charging circuit operates in two modes to balance energy throughput with noise reduction. When plugged into an alternating current (AC) outlet or other external source with a power cable, the wireless charger uses a more sinusoidal wave for wireless power transfer. When disconnected from the external source and running on battery power, the wireless charging circuit can switch to a more square wave to maximize power throughput. As a significant advantage, this approach uses the sine wave to reduce harmonics and high-frequency components in configurations where a cable is present that may act as an antenna for RF noise. Conversely, when no external cable is present—and thus no unintended antenna—the system can use alternative waveforms, such as a square wave, to increase power throughput without radiating high frequency artifacts.

### RELATED APPLICATIONS

This application claims the benefit of U.S. Prov. App. No. 63/801,590, filed on May 7, 2025, the entire content of which is hereby incorporated by reference.

### BACKGROUND

An internal battery for a consumer device can be wirelessly recharged through a wireless power transfer interface between a recharging power source and the device. However, the magnitude and efficiency of power transfer depend on the power transmission waveform. There remains a need for a wireless charging system that optimally adapts to various recharging contexts, such as recharging from a battery and recharging from an AC line.

### SUMMARY

A wireless charging circuit operates in two modes to balance energy throughput with noise reduction. When plugged into an alternating current (AC) outlet or other external source with a power cable, the wireless charger uses a sinusoidal wave for wireless power transfer. When disconnected from the external source and running on battery power, the wireless charging circuit can switch to a more square wave to maximize power throughput. As a significant advantage, this approach uses the sine wave to reduce harmonics and high-frequency components in configurations where a cable is present that may act as an antenna for RF noise. Conversely, when no external cable is present—and thus no unintended antenna—the system can use alternative waveforms, such as a square wave, to increase power throughput without radiating high-frequency artifacts.

In one aspect, a charging system disclosed herein may include a wireless charging antenna; a wireless charging circuit coupled to the wireless charging antenna and configured to transmit a wireless power signal based on a charging waveform from the wireless charging antenna to a wireless power receiving antenna; an internal power source coupled to the wireless charging circuit; a connector and power circuitry for coupling the wireless charging circuit to an external power source; and a controller, the controller configured to perform the steps of: detecting an internal power mode when the connector is uncoupled from the external power source, in response to detecting the internal power mode, providing the charging waveform for driving the wireless charging circuit based on a first waveform, detecting an external power mode when the connector is coupled to the external power source, and in response to detecting the external power mode, providing the charging waveform for driving the wireless charging circuit based on a second waveform different than the first waveform.

Detecting the external power mode may include detecting the external power mode when the connector may be receiving power from the external power source. The connector may include a cable and an alternating current plug. The power circuitry may include an AC/DC converter. The connector may include a cable and a Universal Serial Bus connector. The power circuitry may include a DC/DC converter.

In another aspect, a system disclosed herein includes a wireless charging circuit coupled to a wireless charging antenna and configured to transmit a wireless power signal from the wireless charging antenna to a wireless power receiving antenna based on a charging waveform from the wireless charging circuit; a circuit for detecting a transition between an internal power source for the wireless charging circuit and an external power source for the wireless charging circuit; and a controller configured to respond to the transition between the internal power source and the external power source by switching between a first waveform for driving the wireless charging circuit and a second waveform for driving the wireless charging circuit.

The controller may switch to the first waveform in response to the wireless charging circuit transitioning to the internal power source and switches to the second waveform in response to the wireless charging circuit transitioning to the external power source. The first waveform for the internal power source may include a square wave. The second waveform for the external power source may include a sine wave. The second waveform may have fewer harmonics than the first waveform. The second waveform may have fewer high frequency components than the first waveform. The second waveform may have greater spectral concentration than the first waveform. The first waveform may have greater spectral dispersion than the second waveform. The system may include a connector for removably and replaceably coupling the wireless charging circuit to the external power source. Detecting the transition may include detecting when the connector may be connected to or disconnected from the external power source.

In another aspect, a method disclosed herein includes driving a wireless charging circuit with a charging waveform to wirelessly transmit power; detecting a transition between an internal power source for operating the wireless charging circuit and an external power source for operating the wireless charging circuit, wherein the external power source provides power to the wireless charging circuit through a cable; and in response to detecting the transition, switching between a first waveform for driving the wireless charging circuit and a second waveform for driving the wireless charging circuit, wherein the first waveform has a greater spectral dispersion than the second waveform.

The second waveform may have fewer harmonics than the first waveform. The second waveform may have fewer high frequency components than the first waveform.

In another aspect, a computer program product disclosed herein includes computer-executable code stored in a non-transitory computer-readable medium that, when executing on one or more computing devices, causes the one or more computing devices to perform the steps of: driving a wireless charging circuit with a charging waveform to wirelessly transmit power; detecting a transition between an internal power source for operating the wireless charging circuit and an external power source for operating the wireless charging circuit, wherein the external power source provides power to the wireless charging circuit through a cable; and in response to detecting the transition, switching between a first waveform for driving the wireless charging circuit and a second waveform for driving the wireless charging circuit, wherein the first waveform has a greater spectral dispersion than the second waveform.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, with emphasis instead being placed upon illustrating the principles of the devices, systems, and methods described herein. In the drawings, like reference numerals generally identify corresponding elements.

FIG. 1 shows a physiological monitoring device.

FIG. 2 shows a physiological monitoring system.

FIG. 3 shows a sensing system.

FIG. 4A shows examples of physiological monitoring devices.

FIG. 4B shows examples of physiological monitoring devices.

FIG. 4C shows examples of physiological monitoring devices.

FIG. 5 shows a smart garment system.

FIG. 6 shows a wireless charging system.

FIG. 7 shows alternative waveforms for driving a wireless charging antenna.

FIG. 8 shows a method for managing wireless power waveforms.

### DESCRIPTION

Embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring device and/or system.

The term “continuous,” as used herein in connection with heart rate data, refers to the acquisition of heart rate data at a sufficient frequency to enable detection of individual heartbeats, and also refers to the collection of heart rate data over extended periods, such as an hour, a day or more (including acquisition throughout the day and night), etc. More generally, with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics, such as heart rate, heart rate variability, heart rate peak detection, pulse shape, and so forth. Continuous monitoring should also be understood to include periodic sampling at any suitable interval, duration, and frequency. Thus, for example, continuous monitoring may include measuring a user's body temperature once every ten minutes, or monitoring heart activity by alternately sampling the heart rate for a minute and then pausing sampling for a minute, e.g., to conserve power or memory at times when the measured heart rate indicates that the user is at rest. Sampling may also be dynamic based on sensor input, for example, increasing the sampling rate when signal variability increases, or during periods of relatively higher motion, or based on user input.

At the same time, continuous monitoring is not intended to exclude ordinary data acquisition interruptions, such as temporary displacement of monitoring hardware due to sudden movements, changes in external lighting, loss of electrical power, physical manipulation and/or adjustment by a wearer, physical displacement of monitoring hardware due to external forces, and so forth. It will also be noted that heart rate data or a monitored heart rate, in this context, may more generally refer to raw sensor data, such as optical intensity signals, or processed data therefrom, such as heart rate data, signal peak data, heart rate variability data, or any other physiological or digital signal suitable for recovering heart rate information as contemplated herein. Furthermore, such heart rate data may generally be captured over some historical period that can be subsequently correlated to various other data or metrics related to, e.g., sleep states, recognized exercise activities, resting heart rate, maximum heart rate, and so forth.

The term “computer-readable medium,” as used herein, refers to any non-transitory storage media, such as storage hardware, storage devices, computer memory that may be accessed by a controller, a microcontroller, a microprocessor, a computational system, or the like, or any other module or component of a computational system to encode thereon computer-executable instructions, software programs, and/or other data. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), virtual or physical computer system memory, physical memory hardware, such as random access memory (such as DRAM, SRAM, EDO RAM), and so forth. Although not depicted, any of the devices or components described herein may include a computer-readable medium or other memory for storing program instructions, data, and the like.

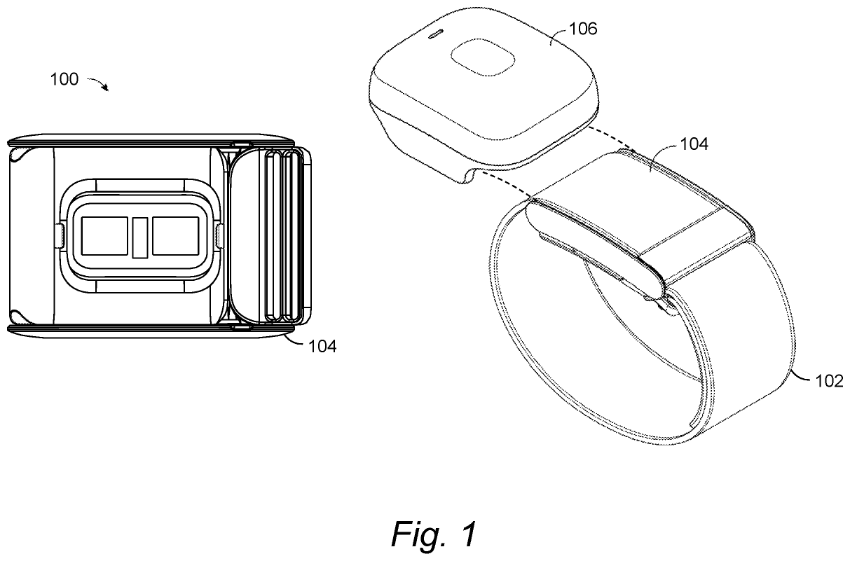

FIG. 1 shows a physiological monitoring system. The system 100 may include a wearable monitor 104 that is configured for physiological monitoring. The system 100 may also include a removable and replaceable battery 106 for recharging the wearable monitor 104. The wearable monitor 104 may include a strap 102 or other retaining system(s) for securing the wearable monitor 104 in a position on a wearer's body for the acquisition of physiological data as described herein. For example, the strap 102 may include a slim elastic band formed of any suitable elastic material such as rubber or a woven polymer fiber such as a woven polyester, polypropylene, nylon, spandex, and so forth. The strap 102 may be adjustable to accommodate different wrist sizes, and may include any latches, hasps, or the like to secure the wearable monitor 104 in an intended position for monitoring a physiological signal. While a wrist-worn device is depicted, it will be understood that the wearable monitor 104 may be configured for positioning in any suitable location on a user's body, based on the sensing modality and the nature of the signal to be acquired. For example, the wearable monitor 104 may be configured for use on a wrist, a forearm, an ankle, a lower leg, a bicep, a chest, side torso, back, a gluteus, behind the ear, forehead, or any other suitable location(s), and the strap 102 may be, or may include, a waistband or other elastic band or the like within an article of clothing or accessory. In another aspect, the wearable monitor 104 may be configured as a ring, earring, stick-on, clip-on, head-mounted (e.g., glasses or goggles), or other article of clothing or accessory that can be worn by a user, and that contains suitable instrumentation, memory, and/or processing for physiological monitoring as described herein. The wearable monitor 104 may also or instead be structurally configured for placement on or within a garment, e.g., permanently or in a removable and replaceable manner. To that end, the wearable monitor 104 may be shaped and sized for placement within a pocket, slot, and/or other housing that is coupled to or embedded within a garment. In such configurations, the pocket or other retaining arrangement on the garment may include sensing windows or the like so that the wearable monitor 104 can operate while placed for use in the garment. U.S. Pat. No. 11,185,292 and U.S. Pat. Pub. No. 2024/0106283 describe non-limiting example embodiments of suitable wearable monitors 104, and are incorporated herein by reference in their entirety. And while the present disclosure may refer to a wrist-worn wearable or other wearable, it should be understood that any of the other locations or forms described herein are also included unless expressly stated to the contrary or otherwise clear from the context.

The system 100 may include any hardware components, subsystems, and the like to support various functions of the wearable monitor 104, such as data collection, processing, display, and communications with external resources. For example, the system 100 may include hardware for a heart rate monitor using, e.g., photoplethysmography, electrocardiography, or any other technique(s). The system 100 may be configured such that, when the wearable monitor 104 is placed for use about a wrist (or at some other body location), the system 100 initiates acquisition of physiological data from the wearer. In some embodiments, the pulse or heart rate may be acquired optically based on a light source (such as light-emitting diodes (LEDs)) and optical detectors in the wearable monitor 104. The LEDs may be positioned to direct illumination toward the user's skin, and optical detectors such as photodiodes may be used to capture illumination intensity measurements indicative of illumination from the LEDs that is reflected and/or transmitted by or through the wearer's skin, or depending on the configuration, through capillaries or arteries.

The system 100 may be configured to record other physiological and/or biomechanical parameters, including, but not limited to, skin temperature (using a thermometer), galvanic skin response (using a galvanic skin response sensor), motion (using one or more multi-axis accelerometers and/or gyroscopes), blood pressure (via physical pressure measurements or other means), sound, electrocardiograms, and the like, as well as environmental or contextual parameters such as ambient light, ambient temperature, humidity, time of day, location, and so forth. For example, the wearable monitor 104 may include sensors such as accelerometers and/or gyroscopes for motion detection, sensors for environmental temperature sensing, sensors to measure electrodermal activity (EDA), sensors to measure galvanic skin response (GSR) sensing, and so forth. The system 100 may also or instead include other systems or subsystems supporting additional functions of the wearable monitor 104. For example, the system 100 may include communications systems to support, e.g., near-field communications, proximity sensing, touch sensing (e.g., via capacitive or resistive sensors), Bluetooth communications, Wi-Fi communications, cellular communications, satellite communications, and so forth. The wearable monitor 104 may also or instead include components such as a Global Positioning System (GPS), a display and/or user interface, a clock and/or timer, and so forth.

The wearable monitor 104 may include one or more sources of battery power, such as a first battery within the wearable monitor 104 and a second battery 106 that is removable from and replaceable to the wearable monitor 104 in order to recharge the battery in the wearable monitor 104. The wearable monitor 104 may also or instead include systems for energy harvesting via, e.g., kinetic energy capture, ambient electromagnetic radiation capture, solar/optical energy capture, and so forth, as well as systems for short and/or medium-range wireless energy transfer to receive power from nearby wireless power sources. Also or instead, the system 100 may include a plurality of wearable monitors 104 (and/or other physiological monitors) that can share battery power or provide power to one another, e.g., using a garment power infrastructure, wireless power sharing network, or the like. The system 100 may perform numerous functions related to continuous monitoring, such as automatically detecting when the user is asleep, awake, exercising, and so forth, and such detections may be performed locally at the wearable monitor 104 or at a remote service such as a mobile device or cloud computing resource coupled in a communicating relationship with the wearable monitor 104 and receiving data therefrom. In general, the system 100 may support continuous, independent monitoring of a physiological signal such as a heart rate, and the underlying acquired data may be stored on the wearable monitor 104 for an extended period until it can be uploaded to a remote processing resource for more computationally complex analysis. In one aspect, the wearable monitor 104 may be a wrist-worn photoplethysmography device, although other form factors are also or instead possible as described herein, such as a ring, a bicep band, a calf band, an elastic band in a garment, a patch, a clip-on device, and so forth.

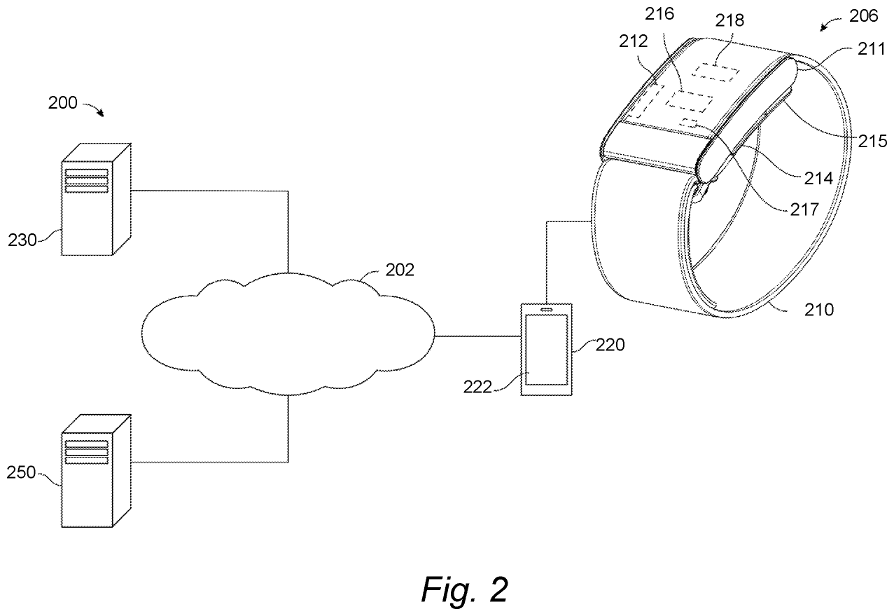

FIG. 2 illustrates a physiological monitoring system. More specifically, FIG. 2 illustrates a physiological monitoring system 200 that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short-range communications infrastructure suitable, e.g., for coupling the physiological monitor 206 to the user device 220, or otherwise supporting communication with local resources. By way of non-limiting examples, short-range communications may include Wi-Fi communications, Bluetooth communications, infrared communications, near-field communications, communications with RFID tags or readers, and so forth.

The physiological monitor 206 may, in general, be any physiological monitoring device or system, such as any of the wearable monitors or other monitoring devices or systems described herein. In one aspect, the physiological monitor 206 may be a wearable physiological monitor shaped and sized to be worn on a wrist or other body location. The physiological monitor 206 may include a wearable housing 211, a network interface 212, one or more sensors 214, one or more light sources 215, a processor 216, a haptic device 217 or other user input/output hardware, a memory 218, and a strap 210 for retaining the physiological monitor 206 in a desired location on a user. In one aspect, the physiological monitor 206 may be configured to acquire heart rate data and/or other physiological data from a wearer in an intermittent or substantially continuous manner. In another aspect, the physiological monitor 206 may be configured to support extended, continuous acquisition of physiological data, e.g., for several days, a week, or more.

In one aspect, the physiological monitor 206 may be configured to continuously or intermittently acquire, store, and/or process physiological data at a frequency sufficient to resolve cardiac activity, including a heart rate and heart rate variability. This may, for example, usefully include sampling rates of 50 Hz (20 ms) or greater on one or more optical channels, as well as synchronized acquisition of temperature data, motion data, and the like. As a significant advantage, this rate of data acquisition can facilitate real-time or near real-time delivery of metrics such as any of the physiological metrics described herein, as well as accurate peak detection, resolution of heart rate variability (typically reported in milliseconds), and extraction of other cardiac metrics and so forth. Similarly, the availability of computational resources, either local or remote, to perform signal processing, embedding, foundation model inferencing, and the like can support real-time coaching or the like to enable timely, actionable user feedback based on the data acquired with the physiological monitor.

The network interface 212 of the physiological monitor 206 may be configured to couple the physiological monitor 206 to one or more other components of the system 200 in a communicating relationship, either directly, e.g., through a cellular data connection or the like, or indirectly through a short-range wireless communications channel coupling the physiological monitor 206 locally to a wireless access point, router, computer, laptop, tablet, cellular phone, or other device that can locally process data, and/or relay data from the physiological monitor 206 to the remote server 230 or other resource(s) 250 as necessary or helpful for acquiring and processing data from the physiological monitor 206. The network interface 212 may also or instead facilitate connections among multiple wearable devices, power sources, and the like, e.g., in a wearable device area network or other multi-device monitoring infrastructure.

The one or more sensors 214 may include any of the sensors described herein, or any other sensors or sub-systems suitable for physiological monitoring or supporting functions. By way of example and not limitation, the one or more sensors 214 may include one or more of a light source (including, e.g., LEDs or other wavelength-specific sources of green light, red light, infrared light, and so forth, as well as broadband illumination), an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, and so forth. The one or more sensors 214 may also or instead include sensors (and accompanying hardware/software) for, e.g., a Global Positioning System, a proximity sensor, an RFID tag reader, an RFID tag, a temporal sensor, an electrodermal activity sensor, an electrocardiogram, a pressure sensor, an acoustic sensor (e.g., a microphone), a camera (e.g., visible light and/or infrared), and the like. The one or more sensors 214 may be disposed in the wearable housing 211, or otherwise positioned and configured for physiological monitoring or other functions described herein. In one aspect, the one or more sensors 214 include a light detector configured to provide light intensity data to the processor 216 (or to the remote server 230) for calculating a heart rate and a heart rate variability. The one or more sensors 214 may also or instead include an accelerometer, gyroscope, and the like configured to provide motion data to the processor 216, e.g., for detecting activities such as a sleep state, a resting state, a waking event, exercise, and/or other user activity. In an implementation, the one or more sensors 214 may include a sensor to measure a galvanic skin response of the user. The one or more sensors 214 may also or instead include electrodes or the like for capturing electronic signals, e.g., to obtain an electrocardiogram and/or other electrically-derived physiological measurements.

The processor 216 and memory 218 may be any of the processors and memories described herein. In one aspect, the memory 218 may store physiological data obtained by monitoring a user with the one or more sensors 214, and/or any other sensor data, program data, or other data useful for operation of the physiological monitor 206 or other components of the system 200. It will be understood that, while only the memory 218 on the physiological monitor is illustrated, any other device(s) or components of the system 200 may also or instead include a memory to store program instructions, raw data, processed data, user inputs, and so forth. In one aspect, the processor 216 of the physiological monitor 206 may be configured to obtain heart rate data from the user, such as heart rate data including or based on the raw data from the sensors 214. The processor 216 may also or instead be configured to determine, or assist in a determination of, a condition of the user related to, e.g., health, fitness, strain, recovery, sleep, or any of the other conditions described herein.

The one or more light sources 215 may be coupled to the wearable housing 211 and controlled by the processor 216. At least one of the light sources 215 may be directed toward the skin of a user adjacent to the wearable housing 211. Light from the light source 215, or more generally, light at one or more wavelengths of the light source 215, may be detected by one or more of the sensors 214, and processed by the processor 216 as described herein.

The system 200 may further include a remote data processing resource executing on a remote server 230. The remote data processing resource may include any of the processors and related hardware described herein, and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep state, exercise activity, exercise type, sleep quality, daily activity strain, and any other health or fitness conditions that might be detected based on such data.

The system 200 may include one or more user devices 220, which may work together with the physiological monitor 206, e.g., to provide a display, or more generally, user input/output, for user data and analysis, and/or to provide a communications bridge from the network interface 212 of the physiological monitor 206 to the data network 202 and the remote server 230. For example, the physiological monitor 206 may communicate locally with a user device 220, such as a smartphone of a user, via short-range communications, e.g., Bluetooth, or the like, for the exchange of data between the physiological monitor 206 and the user device 220, and the user device 220 may in turn communicate with the remote server 230 via the data network 202 in order to forward data from the physiological monitor 206 and to receive analysis and results from the remote server 230 for presentation to the user. In one aspect, the user device(s) 220 may support physiological monitoring by processing or pre-processing data from the physiological monitor 206 to support extraction of heart rate or heart rate variability data from raw data obtained by the physiological monitor 206. In another aspect, computationally intensive processing may advantageously be performed at the remote server 230, which may have greater memory capabilities and processing power than the physiological monitor 206 and/or the user device 220.

The user device 220 may include any suitable computing device(s) including, without limitation, a smartphone, a desktop computer, a laptop computer, a network computer, a tablet, a mobile device, a portable digital assistant, a cellular phone, a portable media or entertainment device, or any other computing devices described herein, including, e.g., supplemental wearable devices and/or computers. The user device 220 may provide a user interface 222 for access to data and analysis by a user, and/or to support user control of operation of the physiological monitor 206. The user interface 222 may be maintained by one or more applications executing locally on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform physiological and/or health monitoring/analyses or any of the other analyses described herein, (e.g., analyzing sleep, determining strain, assessing recovery, and so on), and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The system 200 may include other resources 250, such as any resources that can be usefully employed in the devices, systems, and methods as described herein. For example, these other resources 250 may include other data networks, databases, processing resources, cloud data storage, data mining tools, computational tools, data monitoring tools, algorithms, and so forth. In another aspect, the other resources 250 may include one or more administrative or programmatic interfaces for human actors such as programmers, researchers, annotators, editors, analysts, coaches, and so forth, to interact with any of the foregoing. The other resources 250 may also or instead include any other software or hardware resources that may be usefully employed in the networked applications as contemplated herein. For example, the other resources 250 may include payment processing servers or platforms used to authorize payment for access, content, or option/feature purchases. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200 such as firmware upgrades, user interfaces, and storage and/or pre-processing of data from the physiological monitor 206 before transmission to the remote server 230.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may, for example, include or provide a user interface 222 for web access to the remote server 230 or a database or other resource(s) to facilitate user interaction through the data network 202, e.g., from the physiological monitor 206 or the user device 220.

In another aspect, the other resources 250 may include fitness equipment or other fitness infrastructure. For example, a strength training machine may automatically record repetitions and/or added weight during repetitions, which may be wirelessly accessible by the physiological monitor 206 or some other user device 220. More generally, a gym may be configured to track user movement from machine to machine, and report activity from each machine in order to track various strength training activities in a workout. The other resources 250 may also or instead include other monitoring equipment or infrastructure. For example, the system 200 may include one or more cameras to track motion of free weights and/or the body position of the user during repetitions of a strength training activity or the like, and/or the cameras may be integrated into the physiological monitor 206 or other user device 220. Similarly, a user may wear, or have embedded in clothing, tracking fiducials such as visually distinguishable objects for image-based tracking, or radio beacons or the like for other tracking. In another aspect, weights may themselves be instrumented, e.g., with sensors to record and communicate detected motion, and/or beacons or the like to self-identify type, weight, and so forth, in order to facilitate automated detection and tracking of exercise activity with other connected devices.

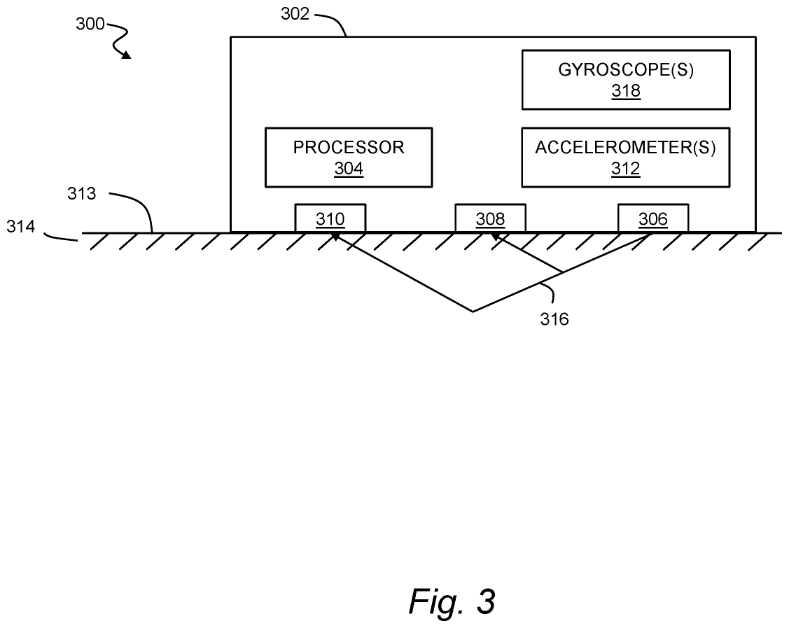

FIG. 3 shows a sensing system. In general, the system 300 may include a physiological monitor 302 with a processor 304, a light source 306, a first sensor 308 (e.g., a first photodetector), a second sensor 310 (e.g., a second photodetector), one or more accelerometers 312, one or more gyroscopes 318, and any other hardware or other components and systems suitable for physiological monitoring as described herein. The physiological monitor 302 may be positioned for use against a surface 313 of the skin 314 of a user where the light source 306 and sensors 308, 310 can contact the skin 314 for acquisition of physiological data. Although not depicted, it will be understood that the physiological monitor 302 may generally be retained in position using any of the straps, garments, patches, bands, clamps, clips, or the like described herein, and/or integrated into other wearable garments, accessories, and the like, such as audio earbuds, earrings or similar, glasses and/or other eyewear, a ring, a headband, and so forth.

The processor 304 may be any microprocessor, microcontroller, application-specific integrated circuit, or other processing circuitry or combination of the foregoing suitable for controlling operation of the physiological monitor and acquiring physiological data.

The light source 306 may include one or more light-emitting diodes or other sources of illumination, and may be positioned within the physiological monitor 302 such that, when the physiological monitor 302 is placed for use on the skin 314, the light source 306 directs illumination toward the skin 314 and the illumination is reflected back toward the sensors 308, 310 as indicated by arrows 316 (or transmitted through the tissue to one or more opposing sensors), where the intensity can be measured. In one aspect, the light source 306 may include light-emitting diodes that emit light in the green, red, infrared, near-infrared, or other suitable wavelength ranges, which can provide desired light transmission through human skin, facilitating low-power transmission of measurable illumination to the sensors 308, 310, although other illumination sources and wavelengths may also or instead be used.

The sensors 308, 310 may be oriented to contact the skin 314 when the physiological monitor 302 is placed for use on this skin 314, and positioned so that the sensors 308, 310 can capture illumination reflected and/or transmitted by the skin from the light source 306. In general, the sensors 308, 310 may include photodiodes, photodetectors, or any other sensor(s) responsive to illumination from the light source 306. This may include broadband optical sensors, narrowband optical sensors, filtered sensors, or the like. In general, a first sensor 308 may be positioned closer to the light source 306 than a second sensor 310 to facilitate detection of differential intensity in the measured wavelength(s). For example, the first sensor 308 may be positioned 1-4 millimeters from the light source 306 and the second sensor 310 may be positioned 2-8 millimeters from the light source, or about twice as far as the first sensor 308 from the light source 306.

Other spacings may also or instead be used depending on, e.g., the intensity of the light source 306, the sensitivity of the sensors 308, 310, the contact force of the physiological monitor 302 on the skin 314, the degree of incursion of ambient light, the physiological measurements/properties of interest, and so forth. In one aspect, the sensors 308, 310 may be linearly arranged in a straight line away from the light source 306. While this provides consistency in comparative measurements, it is not strictly required, and the sensors may be displaced in any of a number of directions away from the light source 306 provided they both contact the skin 314 in a manner that permits capture of light through the skin 314 from the light source 306. In another aspect, the physiological monitor 302 may include one or more other light sources and/or light sensors, which may be arranged to improve accuracy and/or provide redundancy for the contact detection, or to support other measurements such as oxygenation or skin thickness. This may include light sources/sensors using different ranges of wavelengths, different patterns of illumination, and so forth. In another aspect, two sensors may be positioned at different distances from a perimeter of the physiological monitor 302 so that the sensors can acquire differential intensity values for ambient light incident on the skin and transmitted through the skin to the sensors.

In operation, the processor 304 may acquire raw intensity data from the sensors 308, 310, and perform local calculations such as pre-processing raw data for heart rate measurements, or evaluating whether the physiological monitor 302 is properly placed for use on the skin 314.

The accelerometer 312 may include, e.g., one or more single-axis or multi-axis accelerometers, which may usefully measure motion of the physiological monitor 302 to support calculations such as automated activity detection, device on/off evaluation, degree of musculoskeletal activation, and so forth. Other motion and orientation sensing hardware—such as one or more gyroscopes 318, inertial motion sensors, and/or other micro-electromechanical system (MEMS) sensors—may also or instead be used for these purposes. More generally, the physiological monitor 302 may include any additional components, subsystems, and the like suitable for supporting various modes of physiological monitoring and contextual data acquisition as described herein.

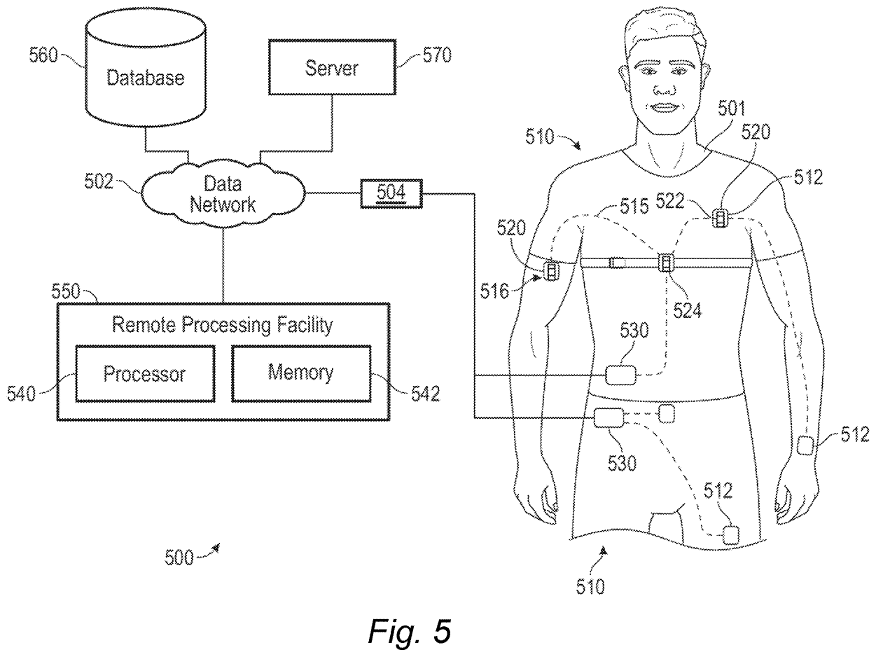

The physiological monitors described herein—e.g., in the systems 100, 200, 300 described above or elsewhere herein—may be provided in one or more different form factors. That is, although a wrist-worn device is illustrated in FIGS. 1 and 2, and garments with sensors are illustrated in FIG. 5, other form factors are also or instead possible, some of which are discussed below by way of example.

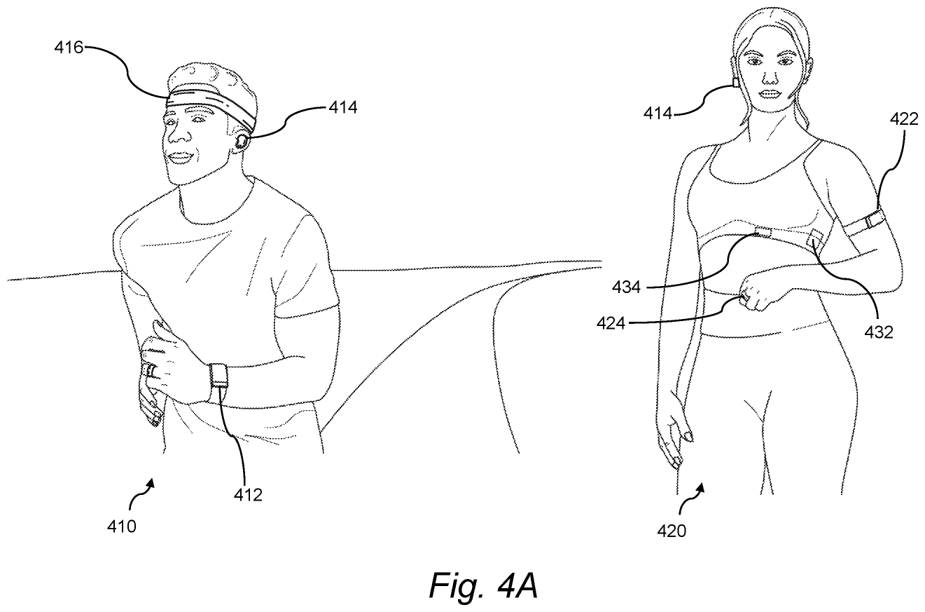

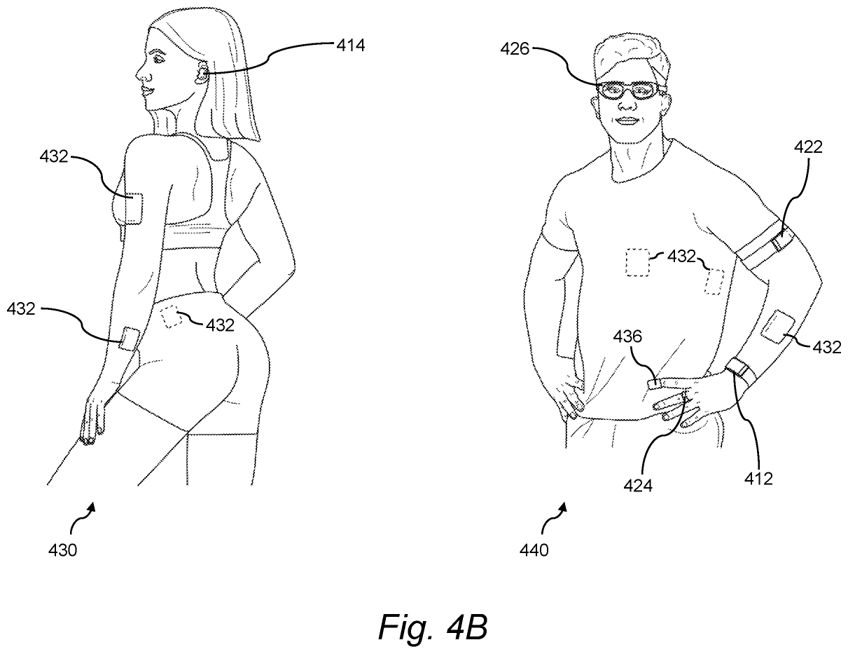

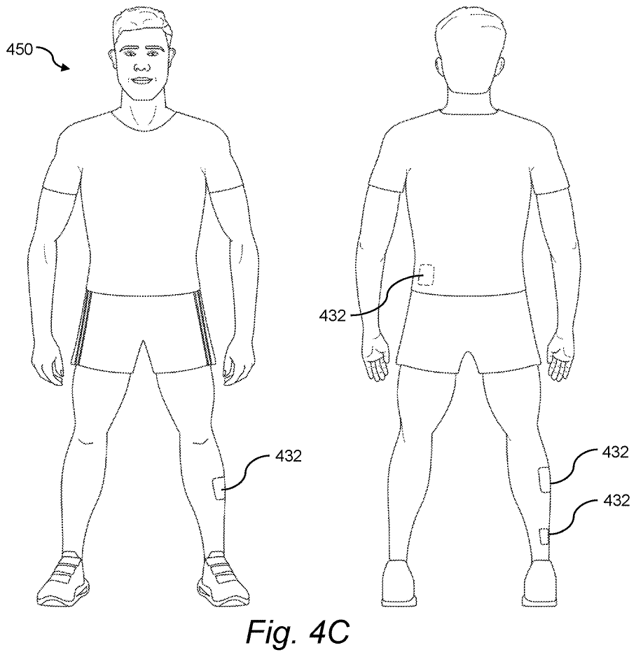

FIGS. 4A-4C illustrate physiological monitoring devices. The illustrated devices may include any of the hardware, software, and/or other components described herein for physiological sensing and/or other functions, and may be embodied in various form factors for various use cases. These various form factors may be used individually or as multiple independent or cooperating physiological monitoring devices, and may include two or more devices of the same type (e.g., two wrist-worn devices, two or more patches, and so on), and/or two or more different types of devices. Moreover, other form factors, and combinations thereof, may also or instead be used for physiological monitoring as described herein. It will further be understood that each of the different example form factors shown in these figures or elsewhere herein may include any one or more of the various sensors, emitters, processors, memories, interfaces, power supplies, and/or other processing and control circuitry, including without limitation any of the foregoing described herein, e.g., with reference to FIGS. 1-3 above.

FIG. 4A shows a first user 410 and a second user 420. The first user 410 may be wearing one or more physiological monitors, such as a wrist-worn device 412 (such as any described herein), an ear-worn device 414 (including on-ear devices retained with a clamp, clip, or other mechanism, and/or in-ear devices, such as earbuds or the like, that are retained at least in part within the ear canal), and a headband 416 or similar.

In one aspect, an ear-worn device 414 may be structurally configured to be partially or entirely inserted within an ear canal of the first user 410. In another aspect, the ear-worn device 414 may be configured to be worn on the ear lobe, or in some other location on the ear where, e.g., temperature, blood flow, respiration, and/or other physiological parameters can be measured. In one aspect, an ear-worn device 414 may be configured for heart rate monitoring, such as any of the heart rate monitoring described herein. For example, this may include continuous heart rate monitoring with optical sensors based on changes in blood volume beneath the skin. The ear-worn device 414 may also or instead be configured for temperature monitoring. For example, the ear-worn device 414 may include one or more infrared sensors, thermistors, thermocouples, or the like to measure the temperature of the ear canal and/or other surfaces. Surface measurements may also or instead be used to support other inferences about body temperature, heat dissipation, and the like, which may be related to current activity levels, general health, and wellness, and so forth.

In another aspect, the ear-worn device 414, or any of the other devices described herein, may be configured for activity tracking. For example, the ear-worn device 414 may include one or more accelerometers, gyroscopes, Global Positioning System (GPS) sensors, and so forth to detect motion and provide information about physical activity levels. This may, for example, include large-scale motion, such as geographical movement and elevation changes that can be tracked with GPS or the like, or local movement detected by the ear-worn device 414, which may be tracked with multi-axis gyroscopes, multi-axis accelerometers, and so forth. These latter sensors may be used to infer, e.g., steps taken, gait analysis, activity type, activity level, and/or overall movement.

The ear-worn device 414, or any of the other devices described herein, may also or instead be configured for blood pressure monitoring. This may, for example, include techniques based on cardiovascular waveform analysis (e.g., using the shape of a PPG or ECG signal from a single location), pulse transit time (e.g., based on the time difference between waveforms at two or more physical locations on the body with two or more monitors), pulse wave velocity (similar to pulse transit time, but over longer arterial distances), physical pulse monitoring (e.g., with pressure sensors, haptic stimulus responses, or other mechanical and/or dynamic techniques), tonometry (measuring the force required to counteract arterial pressure), oscillometric measurement (measuring oscillations in the arterial wall as a cuff deflates around a region of interest), volume clamping (measuring changes in pressure that are required to maintain constant blood volume in a region of interest), and so forth. Some of these blood pressure monitoring techniques are better suited to specific types and locations of monitors and may be more suited to, e.g., wristbands, bicep bands, chest straps, finger rings, and so forth, but are included here for completeness.

The ear-worn device 414, or any of the other devices described herein, may also or instead be configured for electrodermal activity (EDA) monitoring. For example, the ear-worn device 414 may include one or more electrodes in contact with the skin, which may be used to measure the electrical conductance thereof, and to infer, e.g., sweat levels, skin hydration, and/or other parameters correlated to skin conductance. Electrodes may also or instead be used for, e.g., ECG monitoring or the like.

The ear-worn device 414, or any of the other devices described herein, may also or instead be configured to sense blood oxygen saturation (also referred to as pulse oximetry or SpO2) monitoring. To this end, the ear-worn device 414 may include one or more optical sources and detectors, and the system may use different absorption spectra of oxygenated and deoxygenated hemoglobin to estimate pulse oxygen saturation. In another aspect, the ear-worn device 414, or any of the other devices described herein, may be configured for brainwave monitoring, e.g., using electroencephalogram (EEG) sensors to monitor brainwave activity.

The ear-worn device 414, or any of the other devices described herein, may also or instead be configured for respiration rate monitoring. In one aspect, respiration rate may be inferred using respiratory sinus arrhythmia or other techniques to infer respiration rate from a measured heart rate signal over time. In another aspect, respiration rate may be inferred from physical changes in the ear canal (or chest, or other body part, where applicable to a particular sensor). Other techniques may also or instead be used. For example, the ear-worn device 414 may include a microphone or other audio transducer, and the respiration rate may be inferred from audio data acquired from the user.

In another aspect, a headband 416 may be structurally and programmatically configured for physiological sensing and/or monitoring using any of the systems and methods described herein. For example, the headband 416 may be configured to monitor heart rate, temperature, brain activity, electromyography, galvanic skin response, motion, activity, and so forth. In general, the sensors and processing may be adapted for the form factor of the headband 416. For example, the headband 416 may use temperature sensors to measure skin temperature and/or ambient temperature around the head. For brain activity, the headband 416 may include EEG sensors or the like embedded within the headband 416 to measure electrical activity in the brain, which can be used for monitoring brain waves associated with different states such as relaxation, concentration, and/or sleep. More generally, any physiological monitoring techniques described herein that can be adapted for use in a corresponding form factor may be deployed, either alone or in combination, for physiological monitoring with the headband 416. In another aspect, the headband 416 may incorporate a brain-computer interface (BCI) for control of a physiological monitoring system. This may, for example, include any system suitable for direct communication between the brain and external devices based on, e.g., signal acquisition using techniques such as electroencephalography, processing of these raw signals, feature extraction and translation, and then command execution based on an inferred user intention.

The second user 420 may be wearing one or more physiological monitors such as an ear-worn device 414 (which may be any as described herein, and which may be configured as a clamp, clip, earring, or similar, as shown), a bicep band 422, a ring 424, a patch 432 (such as any as described herein, e.g., with reference to FIG. 4B), and a band sensor 434.

The bicep band 422 may be configured for physiological monitoring and sensing using any of the systems and methods described herein, e.g., by retaining a sensor in place with the bicep band 422 or integrating components of the sensor into the bicep band 422, or some combination of these. The bicep band 422 may be configured to monitor heart rate, motion, activity, temperature, blood pressure, blood oxygen saturation, hydration, body composition, ultraviolet light exposure, electrodermal activity, and so forth, as well as combinations of the foregoing. In one aspect, electromyography (EMG) may be used to measure electrical activity in the muscles, e.g., with one or more electrical contacts or the like embedded in the bicep band 422, which can provide information about muscle contraction and fatigue during physical activity. Body composition analysis may be performed using, e.g., bioelectrical impedance analysis to estimate various components of body composition such as fat (percentage or mass), muscle (percentage or mass), and hydration. In another aspect, the bicep band 422 may include one or more sensors to measure ambient light, and more specifically, ambient ultraviolet (UV) light. This may be used to monitor UV exposure, and to provide recommendations to the user to meet certain healthy thresholds for, e.g., vitamin D synthesis, mood, and immune function, and/or to provide alerts concerning possible overexposure. In another aspect, the bicep band 422 or other form factors described herein may be adapted for gesture control based on the capture of motion signals and corresponding inferences of user intent. While a bicep band 422 is illustrated, it will be understood that similar bands for other body parts may also or instead be used, such as leg bands (or more specifically, thigh bands, calf bands, ankle bands, etc.), chest bands, abdomen bands, neck bands, wristbands, and so forth.

The ring 424 may be configured for physiological monitoring and sensing using any of the systems and methods described herein. For example, the ring 424 may be configured to monitor heart rate, motion, activity, sleep, temperature, blood pressure, respiration rate, blood oxygen saturation, hydration, UV exposure, and so forth. A ring 424 is also advantageously positioned to capture a wide range of hand motions, and may be configured for gesture control of physiological monitoring and/or related hardware and software. The ring 424 may be configured for wearing on a finger, as shown in the figure, or another portion of a wearer's body (e.g., a thumb, a toe, and so forth).

The band sensor 434 may be the same or similar to the other monitors described herein and/or any of the bands as described herein. In an aspect, the band sensor 434 may include a monitor inserted into (e.g., placed into a pocket or the like), coupled with, embedded within, or the like, a strap or band, e.g., an elastic band in an article of clothing, an accessory, or similar.

FIG. 4B shows a third user 430 and a fourth user 440. The third user 430 may be wearing one or more physiological monitors such as an ear-worn device 414, which may be the same as or similar to any of those described herein, and one or more patches 432 that include sensors and the like to support physiological monitoring. By way of example, a patch 432 may be configured for physiological monitoring and sensing of heart rate monitoring, temperature, activity, motion, blood pressure, blood oxygen saturation, respiration rate, blood glucose, perspiration, hydration, ultraviolet exposure, and so forth, as well as combinations of the foregoing. In one aspect, the patch 432 may include a continuous glucose monitor with a sensor for insertion into fatty tissue under the skin, along with a transmitter to wirelessly transmit glucose data to a smartphone or other device. In another aspect, the patch 432 may include a hydration monitor using, e.g., electrical impedance analysis to measure resistance and reactance of body tissue with a small electrical current, or bioimpedance spectroscopy to measure impedance at various frequencies of electrical current. Hydration monitoring may also or instead use a wearable patch to collect sweat and analyze electrolyte concentrations correlated to hydration. Other techniques for measuring hydration using, e.g., near-infrared spectroscopy or capacitance hygrometry, may also or instead be employed where suitable adaptations can be made to any of the wearable monitors described herein. In another aspect, the patch 432, or any of the other monitors described herein, may be adapted to monitor environmental conditions such as temperature, humidity, air quality, noise, light, and the like that might be used to supplement physiological monitoring when evaluating the condition of a user. In another aspect, the patch 432, or any of the other monitors described herein, may be adapted for electrodermal activity monitoring, e.g., for tracking autonomic nervous system activity, stress, and the like based on galvanic skin response. One or more patches 432 may be coupled to a user in one or more of a plurality of locations on the body, such as those shown on the third user 430—e.g., a portion of an arm (e.g., the upper arm and/or the lower arm), and on or near the gluteus maximus, and similar. Other locations are also or instead possible, such as the chest, the abdomen, the forehead or temples, the wrist, a hand, a finger, a foot, a neck, a backside, the pelvic region, a portion of the back, a portion of a leg, and so forth.

The fourth user 440 may be wearing one or more physiological monitors such as a bicep band 422, a wrist-worn device 412, a ring 424, and a patch 432, which may be the same or similar to any of the monitors described herein. The fourth user 440 further is shown with eyewear 426 and a fingertip monitor 436, as further explained below by way of example.

The eyewear 426 may include sensors or the like in contact areas or similar, such as a temple region, face region (e.g., via the frame or lens), or other head portion of the fourth user 440. For example, the eyewear 426 may be configured for physiological monitoring and sensing of heart rate, temperature, brain activity, motion, activity type, blood pressure, blood oxygen saturation, and so forth, as well as combinations of the foregoing. In one aspect, the eyewear 426 may employ electrooculography (EOG) to measure electrical activity of the muscles around the eyes or another region of the head/face, which can be used, e.g., to track eye movements and provide insights into cognitive states, attention levels, fatigue, and so forth. In another aspect, one or more EEG sensors may be integrated into the frame and/or temples of the eyewear 426 to measure electrical activity in the brain. The eyewear 426 may also or instead be configured to perform eye tracking using cameras and/or infrared or other sensors to monitor movement of the eyes, which can be used for various applications, including human-computer interaction, attention monitoring, and so forth. The eyewear 426 may also or instead be configured for augmented reality (AR) and virtual reality (VR) biometrics, e.g., where the eyewear 426 can include sensors that monitor physiological parameters to enhance user experience and safety, and to visually present information to the user related to any of the foregoing. In another aspect, the eyewear 426 may include cameras, microphones, and the like for recording and tracking environment information.

The fingertip monitor 436 may include a clamp, clip, or the like, and may be the same or similar to any of the physiological monitors described herein. In some aspects, the fingertip monitor 436 may include a pulse oximeter configured to measure oxygen saturation and/or heart rate for monitoring respiratory and/or cardiovascular health.

FIG. 4C shows the front and back of a fifth user 450 showing further example locations for a patch 432 or the like as described herein.

More generally, any one or more of the sensing modalities described herein may, provided suitable adaptations can be made, be deployed in any one or more of the wearable devices described herein. Furthermore, one or more of the wearable devices may communicate with one or more other wearable devices and/or with a control device such as a smartphone or other computing device, to perform cooperative monitoring. For example, various monitoring techniques, such as electrocardiography or blood pressure measurements using pulse transit time, may usefully be performed by combining signals from sensors at two or more different body locations, and a control device may usefully acquire signals from multiple devices and locations to perform such analysis. Similarly, multiple motion signals from different body locations may be used to refine activity detection, measure body temperature, and so forth. Thus, in one aspect, two or more wearable devices may cooperate with one another to perform an integrated sensing operation such as any of those described herein.

In another aspect, any one or more of the wearable electronic devices described herein may use energy harvesting to generate power from various external sources, and/or to supplement power supplied by an internal battery or the like. For example, a device may use solar energy harvesting to extract solar energy from ambient light sources. This may include integrating solar cells or other ambient light collectors into the wearable device to capture energy from sunlight and/or artificial light sources. In another aspect, the device may use kinetic energy harvesting to generate energy from movements by a user of the device. In another aspect, the device may use thermal energy harvesting to generate power based on differences between the body of the wearer and the surrounding environment. The device may also or instead use vibration energy harvesting, radio frequency energy harvesting (e.g., by capturing ambient RF signals, such as Wi-Fi or cellular signals, and converting them into usable electrical power), ambient light harvesting, and so forth. Other techniques may also or instead be used to provide external power, such as beam steering or resonant techniques for short-range or medium-range radio frequency power transfers. More generally, any technique or combination of techniques for powering a device, and/or for supplementing an internal power source such as a battery, with power from ambient sources may be used to power one of the monitoring devices described herein.

FIG. 5 shows a smart garment system. One limitation on wearable sensors can be body placement. Devices are typically wrist-based, and may occupy a location that a user would prefer to reserve for other devices or jewelry, or that a user would prefer to leave unadorned for aesthetic or functional reasons. This location also places constraints on what measurements can be taken, and may also limit user activities. For example, a user may be prevented from wearing boxing gloves while wearing a sensing device on their wrist. To address this issue, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user's body for supporting monitoring hardware, and may further enable sensing techniques that cannot be achieved with single sensing devices. For example, embedding a plurality of physiological sensors or other electronic/communication devices in a shirt may allow electrical sensors to be placed around a torso to support electrocardiogram (ECG) based heart rate measurements, or placed around muscles such as the pectoralis major, latissimus dorsi, biceps brachii, and other major muscle groups to support muscle oxygen saturation measurements. In another aspect, optical sensors may be positioned along an arterial pathway or the like to support pulse transit time measurements for calculation of blood pressure. The infrastructure provided by a garment may also support other supplemental functions beyond physiological monitoring. For example, wireless antennas may be placed above the upper portion of the thoracic spine to achieve desired communications signals, or a contactless payment system may be embedded in a sleeve cuff for interactions with a payment terminal. Smart garments may also free up body surfaces for other devices. For example, if sensors in a wrist-worn device that provide heart rate monitoring and step counting can be instead embedded in a user's undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment that incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications, and so forth. The garment may also or instead include a power supply, a power management system, processing hardware, data storage, and so forth, any of which may support enriched functions for the smart garment.

In general, the smart garment system 500 illustrated in FIG. 5 may include a plurality of components—e.g., a garment 510, one or more modules 520, a controller 530, a processor 540, a memory 542, and so on—capable of communicating with one another over a data network 502. The garment 510 may be wearable by a user 501 and configured to communicate with a module 520 having a physiological sensor 522 that is structurally configured to sense a physiological parameter of the user 501. As discussed herein, the module 520 may be controllable by the controller 530 based at least in part on a location 516 where the module 520 is located on or within the garment 510. This position-based information may be derived from an interaction and/or communication between the module 520 and the garment 510 using various techniques. It will be understood that, while two controllers 530 are shown, the garment 510 may include a single inter-garment controller, or any number of separate controllers 530 in any number of garments 510 (e.g., one per garment, or one for all garments worn by a person, etc.), and/or controllers may be integrated into other modules 520.

For communication over the data network 502, the system 500 may include a network interface 504, which may be integrated into the garment 510, included in the controller 530, or in some other module or component of the system 500, or some combination of these. The network interface 504 may generally include any combination of hardware and software configured to wirelessly communicate data to remote resources. For example, the network interface 504 may use a local connection to a laptop, smartphone, or the like that couples, in turn, to a wide area network for accessing, e.g., web-based or other network-accessible resources. The network interface 504 may also or instead be configured to couple to a local access point such as a router or wireless access point for connecting to the data network 502. In another aspect, the network interface 504 may be a cellular communications data connection for direct, wireless connection to a cellular network or the like.

The data network 502 may be any as described herein. By way of example, some embodiments of the system 500 may be configured to stream information wirelessly to a social network, a data center, a cloud service, and so forth. In some embodiments, data streamed from the system 500 to the data network 502 may be accessed by the user 501 (or other users) via a website. The network interface 504 may thus be configured such that data collected by the system 500 is streamed wirelessly to a remote processing facility 550, database 560, and/or server 570 for processing and access by the user. In some embodiments, data may be transmitted automatically, without user interaction, for example by storing data locally and transmitting the data over available local area network resources when a local access point such as a wireless access point or a relay device (such as a laptop, tablet, or smartphone) is available. In some embodiments, the system 500 may include a cellular system or other hardware for independently accessing network resources from the garment 510 without requiring local network connectivity. It will be understood that the network interface 504 may include a computing device such as a mobile phone or the like. The network interface 504 may also or instead include or be included on another component of the system 500, or some combination of these. Where battery power or communications resources can advantageously be conserved, the system 500 may preferentially use local networking resources when available, and reserve cellular communications for situations where a data storage capacity of the garment 510 is reaching capacity. Thus, for example, the garment 510 may store data locally up to some predetermined threshold for local data storage, below which data is transmitted over local networks when available. The garment 510 may also transmit data to a central resource using a cellular data network only when local storage of data exceeds the predetermined threshold.

The garment 510 may include one or more designated areas 512 for positioning a module to sense a physiological parameter of the user 501 wearing the garment 510. One or more of the designated areas 512 may be specifically tailored for receiving a module 520 therein or thereon. For example, a designated area 512 may include a pocket structurally configured to receive a module 520 therein. Also or instead, a designated area 512 may include a first fastener configured to cooperate with a second fastener disposed on a module 520. One or more of the first fastener and the second fastener may include at least one of a hook-and-loop fastener, a button, a clamp, a clip, a snap, a projection, and a void.

By placing a pocket or the like in one of these designated areas 512, a position of a module 520 can be controlled, and where an RFID tag, sensor, or the like is used, the designated area 512 can specifically sense when a module 520 is positioned there for monitoring, and can communicate the detected location to any suitable control circuitry.

The garment 510 may also or instead incorporate other infrastructure 515 to cooperate with a module 520. For example, the garment infrastructure 515 may include infrastructure 515 related to ECG devices, such as ECG pads (or otherwise electrically conductive sensor pads and/or electrodes that connect to the module 520, controller 530, and/or another component of the system 500), lead wires, and the like. By way of further example, the garment infrastructure 515 may include wires or the like embedded in the garment 510 to facilitate wired data or power transfer between installed modules 520 and other system components (including other modules 520). The infrastructure 515 may also or instead include integrated features for, e.g., powering modules, supporting data communications among modules, and otherwise supporting operation of the system 500. The infrastructure 515 may also or instead include location or identification tags or hardware, a power supply for powering modules 520 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 520, and so forth. More generally, any hardware, software, or combination of these suitable for augmenting operation of the garment 510 and a physiological monitoring system using the garment 510 may be incorporated as infrastructure 515 into the garment 510 as contemplated herein.

The modules 520 may generally be sized and shaped for placement on or within one or more designated areas 512 of the garment 510. For example, in certain implementations, one or more of the modules 520 may be permanently affixed on or within the garment 510. In such instances, the modules 520 may be washable. Also or instead, in certain implementations, one or more of the modules 520 may be removable and replaceable relative to the garment 510. In such instances, the modules 520 need not be washable, although a module 520 may be designed to be washable and/or otherwise durable enough to withstand a prolonged period of engagement with a designated area 512 of the garment 510. A module 520 may be capable of being positioned in more than one of the designated areas 512 of the garment 510. That is, one or more of the plurality of modules 520 may be configured to sense data using a physiological sensor 522 in a plurality of designated areas 512 of the garment 510.

A module 520 may include one or more physiological sensors 522 and a communications interface 524 programmed to transmit data from at least one of the physiological sensors 522. For example, the physiological sensors 522 may include one or more of a heart rate monitor (e.g., one or more PPG sensors or the like), an oxygen monitor (e.g., a pulse oximeter), a blood pressure monitor, a thermometer, an accelerometer, a gyroscope, a position sensor, a Global Positioning System, a clock, a galvanic skin response (GSR) sensor, or any other electrical, acoustic, optical, camera, or other sensor or combination of sensors and the like useful for physiological monitoring, environmental monitoring, or other monitoring as described herein. In one aspect, the physiological sensors 522 may include a conductivity sensor or the like used for electromyography, electrocardiography, electroencephalography, or other physiological sensing based on electrical signals. The data received from the physiological sensors 522 may include at least one of heart rate data and/or similar data related to blood flow (e.g., from PPG sensors), muscle oxygen saturation data, temperature data, movement data, position/location data, environmental data, temporal data, blood pressure data, and so on.

Thus, certain embodiments include one or more physiological sensors 522 configured to provide continuous measurements of heart rate using photoplethysmography or the like. The physiological sensor 522 may include one or more light emitters for emitting light at one or more desired frequencies toward the user's skin, and one or more light detectors for receiving light reflected from the user's skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 522 may also or instead provide at least one of continuous motion detection, environmental temperature sensing, electrodermal activity (EDA) sensing, galvanic skin response (GSR) sensing, and the like.

The system 500 may include different types of modules 520. For example, a number of different modules 520 may each provide a particular function. Thus, the garment 510 may house one or more of a temperature module, a heart rate/PPG module, a muscle oxygen saturation module, a haptic module, a wireless communication module, or combinations thereof, any of which may be integrated into a single module 520 or deployed in separate modules 520 that can communicate with one another. Some measurements, such as temperature, motion, optical heart rate detection, and the like, may have preferred or fixed locations, and pockets or fixtures within the garment 510 may be adapted to receive specific types of modules 520 at specific locations within the garment 510. For example, motion may preferentially be detected at or near extremities, while heart rate data may preferentially be gathered near major arteries. In another aspect, some measurements, such as temperature, may be measured anywhere, but may preferably be measured at a single location in order to avoid certain calibration issues that might otherwise arise through arbitrary placement.

In another aspect, the system 500 may include two or more modules 520 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, blood pressure, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly, where packet loss, latency, and the like may present challenges to real-time processing.

The communications interface 524 may be any as described herein, for example, including any of the features of the network interface 504 described above.

The controller 530 may be configured, e.g., by computer-executable code or the like, to determine a location of the module 520. This may be based on contextual measurements, such as accelerometer data from the module 520, which may be analyzed by a machine learning model or the like to infer a body position. In another aspect, this may be based on other signals from the module 520. For example, signals from sensors, such as photodiodes, temperature sensors, resistors, capacitors, and the like, may be used alone or in combination to infer a body position. In another aspect, the location may be determined based on the proximity of a module 520 to a proximity sensor, RFID tag, or the like at or near one of the designated areas 512 of the garment 510. Based on the location, the controller 530 may adapt operation of the module 520 for location-specific operation. This may include selecting filters, processing models, physiological signal detections, and the like. It will be understood that operations of the controller 530, which may be any controller, microcontroller, microprocessor, or other processing circuitry, or the like, may be performed in cooperation with another component of the system 500, such as the processor 540 described herein, one or more of the modules 520, or another computing device. It will also be understood that the controller 530 may be located on a local component of the system 500 (e.g., on the garment 510, in a module 520, and so on) or as part of a remote processing facility 550, or some combination of these. Thus, in an aspect, a controller 530 is included in at least one of the plurality of modules 520. And, in another aspect, the controller 530 is a separate component of the garment 510, and serves to integrate functions of the various modules 520 connected thereto. The controller 530 may also or instead be remote relative to each of the plurality of modules 520, or some combination of these.

The controller 530 may be configured to control one or more of (i) sensing performed by a physiological sensor 522 of the module 520 and (ii) processing by the module 520 of the data received from a physiological sensor 522. That is, in certain aspects, the combination of sensors in the module 520 may vary based on where it is intended to be located on a garment 510. In another aspect, processing of data from a module 520 may vary based on where it is located on a garment 510. In this latter aspect, a processing resource such as the controller 530 or some other local or remote processing resource coupled to the module 520 may detect the location and adapt processing of data from the module 520 based on the location. This may, for example, include a selection of different models, algorithms, or parameters for processing sensed data.

In another aspect, this may include selecting from among a variety of different activity recognition models based on the detected location. For example, a variety of different activity recognition models may be developed, such as machine learning models, lookup tables, analytical models, or the like, which may be applied to accelerometer data to detect an activity type. Other motion data, such as gyroscope data, may also or instead be used, and activity recognition processes may also be augmented by other potentially relevant data, such as data from a barometer, magnetometer, GPS, and so forth. This may generally discriminate, e.g., between being asleep, at rest, or in motion, or this may discriminate more finely among different types of athletic activity, such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 510, and then use this detected location to select a suitable model for activity recognition. This technique may similarly be applied to calibration models, physiological signal processing models, and the like, or to otherwise adapt processing of signals from a module 520 based on the location of the module 520. In general, determining a location of a module 520 may include, e.g., receiving a sensed location for the module 520, determining the location based on communications between the module 520 and the garment 510, determining the location based on data received from a physiological sensor 522 of the module 520, and so forth.

Once determined using any of the techniques above, the location of a module 520 may be transmitted for storage and analysis to a remote processing facility 550, a database 560, or the like. That is, in addition to the module 520 using this information locally to configure itself for the location in which it is worn, the module 520 may communicate this information to other modules 520, peripherals, or the cloud. Processing this information in the cloud may help an organization determine if a module 520 has ever been installed on a garment 510, which locations are most used, and how modules 520 perform differently in different locations. These analytics may be useful for many purposes and may, for example, be used to improve the design or use of modules 520 and garments 510, either for a population, for a user type, or for a particular user.

As stated above, the system 500 may further include a processor 540 and a memory 542. In general, the memory 542 may bear computer-executable code configured to be executed by the processor 540 to perform processing of the data received from one or more modules 520. One or more of the processor 540 and the memory 542 may be located on a local component of the system 500 (e.g., the garment 510, a module 520, the controller 530, and the like) or as part of a remote processing facility 550 or the like, as shown in the figure. Thus, in an aspect, one or more of the processor 540 and the memory 542 are included on at least one of the plurality of modules 520. In this manner, processing may be performed on a central module or on each module 520 independently. In another aspect, one or more of the processor 540 and the memory 542 are remote relative to each of the plurality of modules 520. For example, processing may be performed on a connected peripheral device, such as a smartphone, laptop, local computer, or cloud resource.

The processor 540 may be configured to assess the quality of the data received from a physiological sensor 522 of the module 520, or otherwise process data as described herein. The memory 542 may store one or more algorithms, models, and supporting data (e.g., parameters, calibration results, user selections, and so forth) and the like for transforming data received from a physiological sensor 522 of the module 520. In this manner, suitable models, algorithms, tuning parameters, and the like may be selected for use in transforming the data based on the location of the module 520 as determined by the controller 530 and/or processor 540 as described herein.

A database 560 may be located remotely and in communication with the system 500 via the data network 502. The database 560 may store data related to the system 500 such as any discussed herein—e.g., sensed data, processed data, transformed data, metadata, physiological signal processing models and algorithms, personal activity history, and the like. The system 500 may further include one or more servers 570 that host data, provide a user interface, process data, and so forth in order to facilitate use of the modules 520 and garments 510 as described herein.

It will be appreciated that the garment 510, modules 520, and accompanying garment infrastructure and remote networking/processing resources may advantageously be used in combination to improve physiological monitoring and achieve modes of monitoring not previously available.

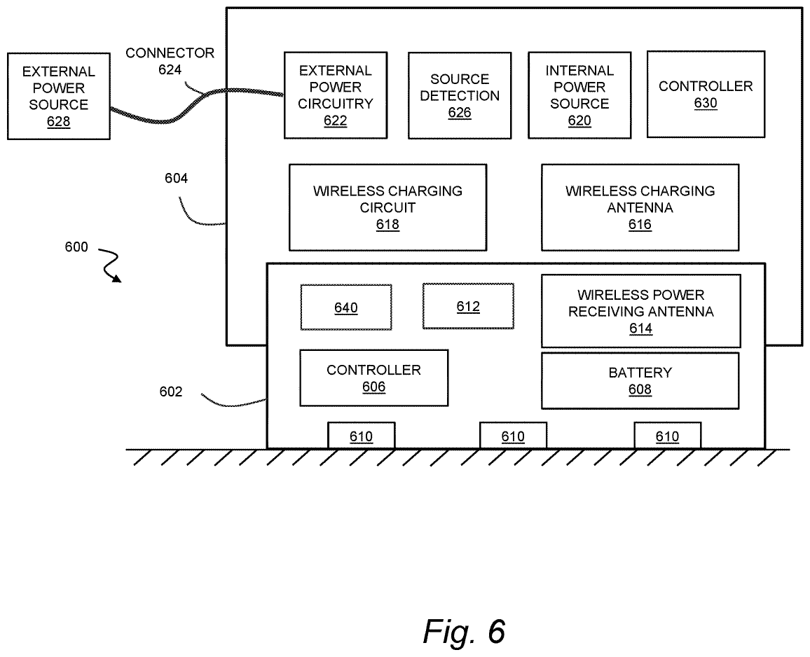

FIG. 6 illustrates a wireless charging system. The charging system 600 may include a wearable device 602, such as any of the wearable devices described herein, and a charger 604.

The charger 604 may be a removable and replaceable battery pack for recharging the wearable device, such as any of the battery packs or the like described herein. The charger 604 may include power circuitry 622 for connecting to an external power source 628, such as an AC line, AC/DC converter, or other external power source or the like, with a connector 624 and configured to convert the outside power source into an electrical power source suitable for use by the controller 630 and other components of the charger 604. This may include providing power to recharge the wearable device 602 through the wireless charging antenna 616 or providing power to recharge the internal power source 620, which can in turn be used to charge the wearable device 602 when the charger 604 is not coupled to an outside power source.

In general, the wearable device 602 may be recharged under various conditions. In one aspect, where the battery pack (e.g., charger 604) is plugged into an external power source, a first waveform may be used to drive the wireless charging antenna 616, such as a sine wave matched to a resonant frequency of the inductive coil of the antenna. When the charger 604 is not connected to an external power source, a second waveform may be used to drive the wireless charging antenna 616, such as a more trapezoidal or square wave. This strategy advantageously minimizes electromagnetic noise transmission through the connector 624 (which may be a long electrical cord that itself operates as an antenna) to prevent noise transmission, comply with EMF transmission regulations, and so forth, when the charger 604 is connected to an external power source. At the same time, this strategy advantageously permits the use of higher-throughput waveforms (for a given peak signal amplitude) such as more trapezoidal or square waves when the power circuitry 622 and/or connector 624 are isolated from the charging circuit. Thus more generally, waveforms may be selected based on the charger 604 connection status to minimize noise while plugged in and maximize power throughput when not plugged in, e.g., in those cases where waveform harmonics can supplement power transmission without transmitting noise through other potential antennae. The controller 630 for the charger 604 may generally operate to detect when the charger 604 is connected to an external power source or otherwise in a state to operate as an antenna for undesirable EMF noise transmissions and when the charger 604 is not connected to an external power source. The controller 630 may then detect when charging is called for (e.g., when the charger 604 is mechanically coupled to the wearable device 602 and positioned for wireless charging) and may select a suitable waveform based on the external connection status.

The charging system 600 may include an external power source 628 such as an alternating current mains supply, a direct current supply, an alternating current/direct current converter, a wall adapter, a power brick, a power supply connected through a cable, a universal serial bus power source, a docking station, a charging cradle, a vehicle power outlet or vehicle power adapter, a portable power bank, a solar power supply, or another external electrical power source suitable for delivering electrical power to the power circuitry 622 through the connector 624. The power circuitry 622 may condition, regulate, rectify, filter, isolate, step up, step down, or otherwise convert power received from the external power source 628 into one or more electrical power forms suitable for operation of the controller 630, the wireless charging circuit 618, and other components of the charger 604. The external power source 628 may also provide power for recharging the internal power source 620 so that the charger 604 can subsequently recharge the wearable device 602 when the charger 604 is disconnected from the external power source 628. It will be understood that the power circuitry 622 may be internal to the charger 604, external to the charger, or some combination of these. For example, the power circuitry 622 may include a power module that couples to an AC main on one hand, converts power to regulated DC, and couples to the charger 604 on the other hand.

In general, the power circuitry 622 may include an AC/DC converter, a DC/DC converter, or any other signal conditioning, power surge protection, electrostatic discharge protection, step-up or step-down conversion circuitry, or other power signal conditioning or the like useful for receiving electrical power from the external power source 628 and converting the electrical power into a form suitable for use in the charger 604.

In the context of this disclosure, the connector 624 may include any hardware or the like for interconnecting the power circuitry 622 of the charger to the external power source 628. In embodiments, the connector 624 may include a plug (such as an alternating current plug for an AC main, or a USB connector(s) for a DC power source), receptacle, cable, cord, lead, terminal, jack, port, contact pad, pogo-pin interface, spring-contact interface, magnetic connector, universal serial bus connector, barrel connector, coaxial power connector, appliance coupler, docking connector, charging-cradle connector, vehicle power connector, or other electrical plug or connector, or other physical, electromechanical interface suitable for coupling the power circuitry 622 to the external power source 628. Portions of the connector 624 may be positioned on or within a housing of the charger 604, may be disposed on a removable cable or adapter, or may be provided as part of an external power module that couples to the charger 604. For example, in one aspect, the connector 624 may include an AC plug, an AC/DC converter for converting AC line power to a DC source such as 5 volts or 9 volts, and a DC or USB plug for connecting the DC output to the charger 604. In some embodiments, the connector 624 may include one or more conductive contacts for power transfer and, optionally, one or more additional contacts or conductors for ground, shielding, source identification, configuration, authentication, temperature sensing, communication, or control. The connector 624 may be keyed, polarized, mechanically latched, magnetically retained, friction-fit, sealed, recessed, shrouded, strain-relieved, or otherwise configured to maintain electrical continuity and reduce unintended disconnection, exposure to contaminants, or misalignment during use. In some embodiments, the connector 624 may include electromagnetic shielding, filtering components, ferrites, a grounded shield, or a cable geometry configured to reduce electromagnetic noise coupling from the wireless charging circuit 618 to the connector 624 or from the connector 624 to the surrounding environment. In the context of the disclosure, the connector 624 may include features such as wires or other electrical leads of sufficient length to act as antennae to radiate energy at radio frequency (RF) wavelengths present in any connected components of the system. While RF noise is a general problem for lengths of conductive material, these RF artifacts may be particularly acute where the length of an electrical lead corresponds to a half wave or integral multiple of an RF component in a signal carried by the connector 624.

The charger 604 may also include an internal power source 620, such as a rechargeable battery or the like, that can alternatively be used to provide power for operating the charger 604 and/or recharging the wearable device 602 through the wireless charging antenna 616. The internal power source 620 may include one or more energy storage devices disposed within or carried by the charger 604. For example, the internal power source 620 may include a rechargeable battery, a lithium-ion battery, a lithium-polymer battery, a solid-state battery, a nickel-metal hydride battery, a thin-film battery, a button cell, a pouch cell, a cylindrical cell, a prismatic cell, a removable battery pack, a replaceable battery pack, a capacitor, a supercapacitor, or combinations thereof. The internal power source 620 may be sized to provide sufficient stored energy to operate the controller 630 and the wireless charging circuit 618 and to recharge the power storage 608 of the wearable device 602 when the charger 604 is not coupled to the external power source 628.

The wireless charging antenna 616 may include an induction coil, a planar coil, a wound-wire coil, a printed conductive trace, a flexible printed circuit coil, a molded interconnect coil, a near field communication antenna, a ferrite-backed coil assembly, a resonant antenna structure, or other wireless power transmission hardware configured to generate a wireless power signal for reception by the power receiving antenna 614 of the wearable device 602. The wireless charging antenna 616 may include one or more turns of conductive material arranged in a circular, oval, rectangular, spiral, solenoidal, or other geometry selected to provide magnetic coupling with the power receiving antenna 614 when the charger 604 is positioned adjacent to or mechanically coupled with the wearable device 602. In some embodiments, the wireless charging antenna 616 may be disposed within a housing of the charger 604, adjacent to a charging surface of the charger 604, in a removable battery pack, in a charging cradle, or in another portion of the charger 604 positioned to align with the power receiving antenna 614 during charging. The wireless charging antenna 616 may also include multiple windings, taps, antenna segments, or switchable coil portions so that the wireless charging circuit 618 can adjust an effective inductance, coupling area, field strength, or operating mode of the wireless charging antenna 616 based on whether power is supplied from the external power source 628 or the internal power source 620, based on alignment with the wearable device 602, or based on a charging state of the power storage 608.

The wireless charging circuit 618 may be integrated into the controller 630, separate from the controller 630, or some combination of these. The wireless charging circuit 618 may include a driver, an inverter, a switching bridge, a resonant tank circuit, an impedance-matching network, a filter, a tuning circuit, a current-sense circuit, a voltage-sense circuit, or combinations thereof. In general, when the circuit 626 for source detection indicates that the charger 604 is coupled to the external power source 628 through the connector 624, the controller 630 may cause the wireless charging circuit 618 to drive the wireless charging antenna 616 with a sinusoidal or more sinusoidal waveform having reduced harmonic content. When the circuit 626 for source detection indicates that the charger 604 is uncoupled from the external power source 628 and operating from the internal power source 620, the controller 630 may cause the wireless charging circuit 618 to drive the wireless charging antenna 616 with a square, trapezoidal, pulsed, or otherwise less sinusoidal waveform that increases power throughput. The controller 630 may also adjust the waveform based on a detected alignment, charging state, received-power indication, temperature, current, voltage, or other operating parameter so that the wireless charging circuit 618 delivers power to the wireless charging antenna 616 at a level suitable for recharging the power storage 608 of the wearable device 602. In one aspect, the wireless charging circuit 618 may receive a waveform from the controller 630, and convert the waveform into a corresponding (e.g., having the same shape) power signal to the wireless charging antenna 616.

The charger 604 may include a controller 630 to manage whether and how power is wirelessly delivered by the wireless charging circuit 618 through the wireless charging antenna 616 to which the wireless charging circuit 618 is connected. The controller 630 may store waveforms for use by the wireless charging circuit 618, or the wireless charging circuit 618 may store a plurality of waveforms and the controller 630 may select from among the plurality of waveforms based on the presence or absence of an external power source 628. The controller 630 may also scale or otherwise modulate the control signal according to charging conditions and so forth. More generally, the controller 630 may cooperate with the wireless charging circuit 618 by generating, selecting, or configuring a charging waveform that is applied by the wireless charging circuit 618 to the wireless charging antenna 616. The controller 630 may determine a charging condition for the charger 604, including whether the charger 604 is operating from the internal power source 620 or from the external power source 628, and may provide one or more control signals to the wireless charging circuit 618 based on the charging condition. The one or more control signals may include an enable signal, a frequency command, a duty-cycle command, an amplitude command, a phase command, a waveform-selection command, a pulse-width modulation signal, a gate-drive control signal, or a digital or analog control value for configuring operation of the wireless charging circuit 618. In response, the wireless charging circuit 618 may convert power received from the internal power source 620 or the power circuitry 622 into an alternating drive signal supplied to the wireless charging antenna 616, thereby causing the wireless charging antenna 616 to generate a time-varying magnetic field or other wireless power signal for reception by the power receiving antenna 614 of the wearable device 602.

The charger 604 may include a circuit 626 for source detection that detects whether the power circuitry 622 is connected to an external power source 628 and that switches between these sources as a power source for use by the controller 630 and wireless charging circuit 618 in delivering wireless power to the wearable device 602. In some embodiments, the circuit 626 for source detection may be implemented within the controller 630, e.g., where the controller 630 includes a microcontroller or other system-on-a-chip circuit that includes suitable analog inputs and outputs. The circuit 626 may also or instead include a voltage detection circuit configured to sense a voltage at the connector 624 or at an input of the power circuitry 622. For example, the circuit 626 for source detection may include a comparator, an analog-to-digital converter input of the controller 630, a threshold detector, a power-good detector, a current-sense circuit, a load-detection circuit, a universal serial bus detection circuit, a rectifier-output sensing circuit, or combinations thereof. In some embodiments, the circuit 626 for source detection may detect and/or identify a connection to the external power source 628 by detecting that an input voltage exceeds a threshold, by detecting current available from the power circuitry 622, by receiving a power-present signal from the power circuitry 622, by sensing a change in impedance at the connector 624, or by detecting a communication or configuration signal associated with the external power source 628. The circuit 626 for source detection may provide a signal to the controller 630 to cause the charger 604 to select power from the external power source 628 when available, select power from the internal power source 620 when the external power source 628 is unavailable, or transition between the external power source 628 and the internal power source 620 while eliminating or reducing interruptions to operation of the wireless charging circuit 618.

In general, the wearable device 602 includes a physical configuration, e.g., flanges, arms, protrusions, clips, and so forth, for removably and replaceably coupling the charger 604 to the wearable device 602, more specifically in an orientation for wireless power transfer therebetween. The wearable device 602 may include any of the wearable devices described herein, and may include components arranged in a configuration to support device functions such as physiological monitoring or the like.

The wearable device 602 may include a device controller 606 to manage device operations. The device controller 606 may include one or more processors, microcontrollers, microprocessors, digital signal processors, application-specific integrated circuits, field-programmable gate arrays, programmable logic devices, state machines, power management controllers, sensor controllers, communication controllers, or combinations thereof. The device controller 606 may be disposed within a housing of the wearable device 602 and may be coupled to one or more components of the wearable device 602, e.g., to manage operation, timing, power consumption, charging, sensing, communication, user interaction, data processing, or combinations thereof.

In some embodiments, the device controller 606 may execute instructions stored in memory 640 of the wearable device 602 to perform one or more device functions. The memory 640 may include volatile memory, non-volatile memory, flash memory, read-only memory, random access memory, electrically erasable programmable read-only memory, registers, cache memory, or combinations thereof. The device controller 606 may receive signals from one or more sensors of the wearable device 602, process sensed data, control one or more output devices, manage wireless communications, store data, retrieve data, or coordinate operation of multiple subsystems of the wearable device 602.

In some embodiments, the device controller 606 may cooperate with the power receiving antenna 614 and associated power receiving circuitry to manage receipt of wireless power from the wireless charging antenna 616 of the charger 604. The device controller 606 may detect that the wearable device 602 is positioned for charging, enable charging of the power storage 608, regulate a charging current or charging voltage, communicate a charging status or received-power indication to the charger 604, or adjust operation of the wearable device 602 during charging. The device controller 606 may also stop, reduce, or modify charging in response to a full-charge condition, an over-temperature condition, an over-voltage condition, an over-current condition, a foreign-object detection condition, misalignment between the wearable device 602 and the charger 604, or another fault condition.

The wearable device 602 may include a power storage 608, such as a rechargeable battery or any of the other power storage components described herein, to provide energy storage capabilities for operation of the wearable device 602.

The wearable device 602 may include a power receiving antenna 614 configured to receive wireless power signals from the charger 604, or more specifically, from the wireless charging antenna 616. The power receiving antenna 614 may include any of the wireless power transfer antennae described herein, or any similar antenna or combination of antennae suitable for wireless power transfer. In general, the power receiving antenna 614 may be physically matched to the wireless charging antenna 616, e.g., with a similar shape, size, and/or resonant frequency. In one aspect, the wireless power connection between the charger 604 and the wearable device 602 may be managed, e.g., with the device controller 606, the controller 630, and/or other wireless power transfer circuitry, to control temperature, power transfer, power efficiency, and so forth.

The wearable device 602 may include multiple sensors 610 and/or other hardware positioned in the wearable device to support, e.g., physiological monitoring as described herein. The wearable device 602 may also include a temperature sensor 612 for monitoring, e.g., an internal temperature of the wearable device 602 or the power storage 608 (or related charging circuitry), or any of the other sensors and related hardware described herein.

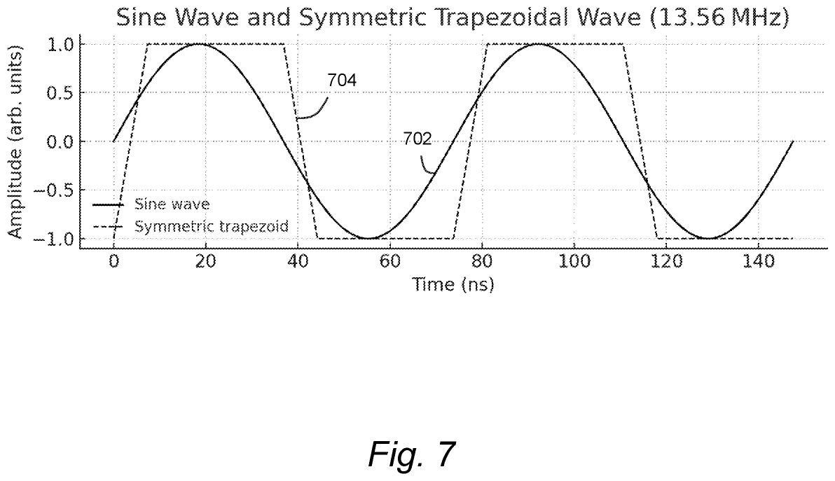

FIG. 7 shows alternative waveforms for driving a wireless charging antenna. It will be understood that a wide range of waveforms can potentially be used for both the connected and disconnected waveforms and may be optimized in various ways for noise minimization and/or power throughput maximization. In one aspect, the connected waveform 702 may be a sine wave such as a sine wave conforming to NFC power transmission standards, and/or matched to a resonant frequency of the induction coils in the wireless charging antenna. By contrast, the disconnected waveform 704, which is used when the battery pack or other charger is not connected to an external power source, may be a square wave, a trapezoidal wave, or some other waveform that adds more spectral components, increases power dispersion, or otherwise facilitates an increase in transmission power.

In a simplified ideal case, a square wave with a peak voltage A provides power over time T of:

E=A²T

By contrast, a sine wave with a peak voltage A provides power over time of T:

$$E = \frac{1}{2} {A}^{2} T$$

Thus, a square can theoretically deliver twice as much energy per unit of time. The square wave may have a lower efficiency of power transmission, particularly where harmonics and other high-frequency components deviate from the resonant frequency of a tuned RF wireless charging circuit or the like. However, this can be a useful design tradeoff where the total power transferred can be increased, notwithstanding the drop in efficiency, where there is sufficient power budget in the alternative power source to accept decreased efficiency, and where the resulting harmonics and other higher frequency components that enable the greater throughput do not create unacceptable electromagnetic noise (e.g., noise exceeding FCC requirements or noise negatively impacting nearby instrumentation).

It should also be appreciated that, while square-like waveforms may have lower efficiency due to harmonic content, they may also improve end-to-end efficiency of a power transfer system under certain conditions. For example, when operating under typical constraints (e.g., voltage/current sources, conversion electronics, wireless power antenna size and placement), increasing power throughput up to a certain threshold can improve overall system performance by improving the efficiency of the power conversion stages on both the charger 604 side and the device 602 side. For example, a transmitter for a battery-powered source typically includes a DC/AC inverter (e.g., an oscillator and H-bridge) and matching network to match the resonant frequency of an inductively coupled wireless interface. Similarly, the receiver may include complementary resonant tuning and an AC/DC rectifier, along with voltage regulation circuitry to stabilize the rectified DC signal. In this context, a more square-like waveform can potentially yield higher effective end-to-end efficiency, even if the wireless transfer segment itself is less efficient, where the operating range yields greater efficiency in the power conversion stages associated with each side of the wireless power transfer interface. In practice, this improved overall efficiency can translate to system-level benefits such as enabling a smaller internal battery in the charger 604 and/or reduced overall system thermal rise in the charger 604 and/or device 602 due to improved power conversion efficiency. Further, the particular waveform (for the more square wave) may be selected or modified based on the resulting efficiency of the power conversion stages, or based on the overall, end-to-end efficiency including power conversion and wireless power transfer.

While square and sine waves usefully illustrate alternative wireless charging strategies, it will be understood that a wide variety of waveforms may also or instead be used to control the amplitude and distribution of spectral content within a wireless power signal, any of which may be used as alternative drive signals as a wireless charging device is connected to and disconnected from external power. For example, the first waveform and the second waveform used to drive wireless power transfer can be characterized by differences in time-domain shape and/or frequency-domain content. A first waveform that is sinusoidal or more sinusoidal has a relatively smooth periodic time-domain shape with relatively gradual transitions between voltage or current levels, a lower slew rate, a lower edge rate, and fewer abrupt changes in amplitude. The sinusoidal or more sinusoidal waveform can concentrate a greater portion of its signal energy in the frequency domain at a base frequency (e.g., a first-harmonic frequency), such as a resonant frequency of the wireless charging antenna 616, a resonant frequency of the power receiving antenna 614, or the resonant frequency of a circuit that includes one or both antennas. By concentrating energy at the base frequency and reducing energy at harmonics of the base frequency, the sinusoidal or more sinusoidal waveform can reduce total harmonic distortion, reduce out-of-band energy, reduce high-frequency spectral components, and reduce corresponding radiated electromagnetic emissions from the connector 624. A waveform of this type may preferably be used to drive wireless charging when the charger 604 is connected to the external power source 628 via the connector 624.

In other embodiments, a second waveform may be square, trapezoidal, pulsed, or otherwise less sinusoidal, such that it has relatively abrupt transitions, a higher slew rate, a higher edge rate, shorter rise times, shorter fall times, and/or one or more corners, steps, discontinuities, or near-discontinuities in the time-domain waveform. These time-domain characteristics correspond to a broader frequency-domain spectrum. For example, a square or more square waveform may contain significantly more and greater amplitude harmonic components at frequencies above the base frequency and/or greater high-frequency content than the sinusoidal or more sinusoidal waveform. This second waveform can achieve higher power transfer at a given peak amplitude by distributing a greater portion of its signal energy across multiple frequency components rather than concentrating the signal energy at the base frequency. The square, trapezoidal, pulsed, or otherwise less sinusoidal waveform can, as a result, be characterized as having greater spectral dispersion (e.g., with frequency domain peaks spread across a greater range of frequency bins), greater occupied bandwidth, greater harmonic-to-base-frequency energy, greater out-of-band energy, greater total harmonic distortion, greater spectral spread, a higher spectral centroid, a slower spectral roll-off, and/or a larger number of spectral components above a threshold amplitude, relative to the sinusoidal or more sinusoidal waveform.

In some embodiments, the controller 630 can select the sinusoidal or more sinusoidal waveform when the charger 604 is coupled to the external power source 628 because the connector 624, a power cable, or another external conductive structure provides a path for conducted noise that operates as an antenna for radiating high-frequency components. Under these conditions, the controller 630 can cause the wireless charging circuit 618 to drive the wireless charging antenna 616 with a waveform having reduced harmonic content, reduced high-frequency components, greater spectral concentration, lower spectral dispersion, smoother transitions, lower slew rate, and/or reduced discontinuity content. Alternatively, the controller 630 can select the square, trapezoidal, pulsed, or otherwise less sinusoidal waveform when the charger 604 is operating from the internal power source 620 because the charger 604 is (or can be) electrically isolated from the external conductive structure of the connector 624. Under these conditions, the controller 630 can permit use of a waveform having higher throughput, broader spectral content, more harmonic components, or greater spectral dispersion.

In some embodiments, the difference between the first waveform and the second waveform can be expressed using one or more quantitative or qualitative waveform parameters. The parameters can include harmonic count, harmonic amplitude, harmonic power, harmonic-to-base-frequency ratio, total harmonic distortion, occupied bandwidth, spectral bandwidth, out-of-band power, high-frequency energy above a threshold frequency, spectral centroid, spectral spread, spectral roll-off, number of zero crossings per unit time, transition time, rise time, fall time, slew rate, edge rate, curvature, continuity, differentiability, peak-to-average ratio, root-mean-square value for a given peak amplitude, or an integrated spectral-energy ratio between a selected frequency band and the base frequency. The second waveform can be selected to have lower values for one or more of the parameters associated with abrupt transitions or high-frequency content, while the first waveform can be selected to have higher values for one or more of the parameters associated with power throughput, harmonic energy, or spectral dispersion.

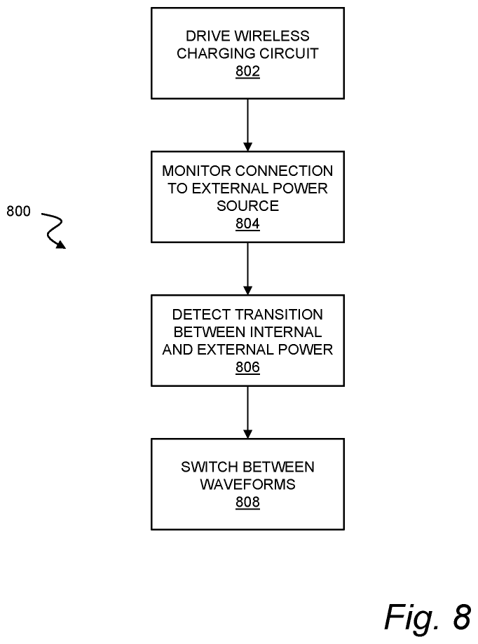

FIG. 8 shows a method for managing wireless power waveforms. As described herein, this may generally include selecting a waveform for wireless power transfer that has a lower spectral dispersion (and lower root-mean square value) when physically connected to an external device and selecting a waveform for wireless power transfer that has a higher spectral dispersion (and higher root-mean square value) when disconnected from the external device.

As shown in step 802, the method 800 includes driving a wireless charging circuit such as any described herein with a charging waveform to wirelessly transmit power. For example, a controller may cause a wireless charging circuit to drive a wireless charging antenna with a charging waveform so that the wireless charging antenna generates a wireless power signal for reception by a power receiving antenna of a wearable device. The charging waveform may be generated using power supplied from an internal power source for a charger providing the charging waveform, or using power supplied from an external power source connected to the charger through a connector. In some embodiments, the charging waveform may include a first waveform or a second waveform, where the charger selects between the first waveform and the second waveform based on a power-source condition of the charger.

As shown in step 804, the method 800 may include monitoring a connection to external power sources for the presence and/or availability of external power, or for the presence of a connector for physically connecting to an external power source. In some embodiments, monitoring for the presence of the connection may include sensing a voltage at an input to power circuitry, e.g., when a sensed voltage exceeds a connection threshold, remains above the connection threshold for a debounce period, satisfies a valid input-voltage range, or causes a power-good signal to be asserted. A circuit for source detection may also or instead determine that the external power source is disconnected when the sensed voltage falls below a disconnection threshold, remains below the disconnection threshold for the debounce period, or causes the power-good signal to be deasserted. In some embodiments, the connection threshold and the disconnection threshold may be different to provide hysteresis and reduce repeated switching when the sensed voltage is near a threshold.

In some embodiments, monitoring for the presence of the connection may include sensing current supplied by the external power source, sensing current available from the power circuitry, sensing current through the connector, or sensing a change in impedance at the connector or at a plug or the like for the connector. For example, the circuit for source detection may include a current-sense resistor, a current mirror, a Hall-effect sensor, a comparator, an analog-to-digital converter input, an impedance-measurement circuit, or a load-detection circuit. The circuit for source detection may also or instead apply a pull-up signal, a pull-down signal, a test current, or a detection pulse to a contact of the connector and may determine whether the external power source or a cable of the connector is present based on a resulting voltage, current, impedance, or timing response.

In some embodiments, monitoring for the presence of the connection may include detecting a configuration signal, identification signal, communication signal, authentication signal, or source-capability signal associated with the external power source. For example, the circuit for source detection or the controller of the charger may monitor one or more contacts of the connector for a resistance value, voltage divider output, digital message, serial communication signal, universal serial bus configuration signal, adapter-identification signal, or other source-identification condition indicating that the external power source is connected and capable of supplying power to the charger. The controller may use the detected condition to distinguish between a mechanically connected cable that is not energized and a connection to an energized external power source, although in either case a suitable waveform may be made if the physical nature of the connected objects can operate as an antenna for RF noise as described herein.

As shown in step 806, the method 800 may include detecting a transition between the internal power source for operating the wireless charging circuit and the external power source for operating the wireless charging circuit. The external power source may include any of the external power sources described herein, and may provide power to the wireless charging circuit through a connector such as a cable and/or other connection hardware. A circuit for source detection may detect a connection of the external power source, a disconnection of the external power source, an availability of power from the external power source, a loss of power from the external power source, a change in a voltage, current, impedance, configuration signal, communication signal, or power-good signal associated with the external power source, or any other change of sensed conditions or control signals indicating a transition between connected and disconnected states. The circuit for source detection may provide a transition signal to the controller for the charger indicating a transition in power sources, based upon which the controller may transition between use of the internal and external power sources and based upon which the controller may transition between a more sinusoidal waveform and a more square waveform according to the detected condition.

In some embodiments, detecting the transition may include filtering or otherwise validating one or more detection signals before identifying the transition. For example, the circuit for source detection (or other circuitry) may debounce the detection signal, average multiple samples, compare the detection signal to multiple thresholds, require the detection signal to satisfy a threshold for a predetermined time, ignore transient spikes, detect brownout or undervoltage conditions, or require both a voltage-present condition and a power-good condition before determining that the external power source is connected or disconnected. The controller may then initiate or identify the associated transition between the internal power source and the external power source and, in response, switch between the first waveform and the second waveform for driving the wireless charging circuit.

As shown in step 808, the method 800 may include, in response to detecting the transition, switching between the first waveform for driving the wireless charging circuit and the second waveform for driving the wireless charging circuit. For example, this may include switching waveforms with the controller, e.g., by causing circuitry of the device to transition to outputting the appropriate one of the waveforms to the antenna according to a current context. In some embodiments, the first waveform may be a more square wave signal or other waveform having a greater spectral dispersion than the second waveform. For example, the first waveform may include a square, trapezoidal, pulsed, or otherwise less sinusoidal waveform having greater harmonic content, greater high-frequency content, a higher slew rate, a faster edge rate, a shorter rise time, a shorter fall time, greater occupied bandwidth, greater total harmonic distortion, or a larger amount of energy distributed among frequency components above a base frequency. The second waveform may include a more sinusoidal waveform having fewer harmonics than the first waveform, fewer high-frequency components than the first waveform, lower spectral dispersion, lower out-of-band energy, lower total harmonic distortion, smoother transitions, a lower slew rate, or a greater portion of energy concentrated at the base frequency. For example, the second waveform may be a sine wave, a band-limited square wave, a soft-edged square wave, or the like.

In an example embodiment, the charger may be connected to an external power source such as an AC main power supply or a USB power supply via a cable and a controller for the charger may use this external power supply for charging a battery and/or generating a wireless power signal to a wearable device or the like connected to the charger. When the cable is disconnected, this change in state may be detected by the controller of the charger, which may initiate a transition to powering the charger with the battery of the charger. The controller may also transition to the battery as a source of power for the wireless power signal to the connected wearable device, and select a waveform with greater spectral dispersion to drive the wireless charging antenna. If the external power source once again becomes available, this may be detected by the circuit where source detection and a corresponding signal may be provided to the controller so that the controller can, in turn, transition to use of the external power supply to power the charger and also transition to a waveform with lower spectral dispersion to drive the wireless charging antenna.

Thus, as described herein, the controller of the charger may switch from the first waveform to the second waveform when a transition indicates that the charger is coupled to the external power source through the connector. The second waveform may advantageously reduce electromagnetic noise radiated by a cable or other physical antenna in the connector. In some embodiments, the controller may switch from the second waveform back to the first waveform when the transition indicates that the charger is disconnected from the external power source and is operating from the internal power source. The first waveform may advantageously increase power transfer, increase power throughput, or permit use of harmonic energy by the wireless charging antenna, the power receiving antenna, or associated resonant circuitry when the charger is not coupled to a parasitic antenna. The switching may include changing a frequency command, duty-cycle command, amplitude command, phase command, pulse-width modulation signal, gate-drive signal, waveform-selection signal, or other control signal provided by the controller to the wireless charging circuit.

A charger may operate continuously in this manner by monitoring for transitions between connected and disconnected states and selecting between corresponding waveforms accordingly. While the initial state is not illustrated in FIG. 8, it will be understood that the system may start in either state (connected or disconnected), or in neither state, and begin with an immediate, initial detection of the connection state before selecting an initial waveform for wireless power transfer. It will also be understood that, while not illustrated, the wireless power circuitry may include substantial additional control logic without deviating from this disclosure, e.g., to detect the presence of a receiving antenna, regulate a power transfer based on temperature or other sensed or controlled conditions, transmit data unidirectionally or bidirectionally through the wireless power interface, and so forth.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application-specific integrated circuits, programmable gate arrays, programmable array logic components, or any other device or devices that may be configured to process electronic signals. It will further be appreciated that a realization of the processes or devices described above may include computer-executable code created using a structured programming language such as C, an object-oriented programming language such as C++, or any other high-level or low-level programming language (including assembly languages, hardware description languages, and database programming languages and technologies) that may be stored, compiled, or interpreted to run on one of the above devices, as well as heterogeneous combinations of processors, processor architectures, or combinations of different hardware and software.

Thus, in one aspect, each method described above, and combinations thereof, may be embodied in computer-executable code that, when executing on one or more computing devices, performs the steps thereof. In another aspect, the methods may be embodied in systems that perform the steps thereof, and may be distributed across devices in a number of ways, or all of the functionalities may be integrated into a dedicated, standalone device or other hardware. The code may be stored in a non-transitory fashion in a computer memory, which may be a memory from which the program executes (such as random access memory associated with a processor), or a storage device such as a disk drive, flash memory, or any other optical, electromagnetic, magnetic, infrared, or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party, such as a remote user, a remote processing resource (e.g., a server or cloud computer), or a machine, to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

What is claimed is:

1. A charging system comprising:
a wireless charging antenna;
a wireless charging circuit coupled to the wireless charging antenna and configured to transmit a wireless power signal based on a charging waveform from the wireless charging antenna to a wireless power receiving antenna;
an internal power source coupled to the wireless charging circuit;
a connector and power circuitry for coupling the wireless charging circuit to an external power source; and
a controller, the controller configured to perform the steps of:
detecting an internal power mode when the connector is uncoupled from the external power source,
in response to detecting the internal power mode, providing the charging waveform for driving the wireless charging circuit based on a first waveform,
detecting an external power mode when the connector is coupled to the external power source, and
in response to detecting the external power mode, providing the charging waveform for driving the wireless charging circuit based on a second waveform different than the first waveform.

2. The charging system of claim 1, wherein detecting the external power mode includes detecting the external power mode when the connector is receiving power from the external power source.

3. The charging system of claim 1, wherein the connector includes a cable and an alternating current plug.

4. The charging system of claim 1, wherein the power circuitry includes an AC/DC converter.

5. The charging system of claim 1, wherein the connector includes a cable and a Universal Serial Bus connector.

6. The charging system of claim 1, wherein the power circuitry includes a DC/DC converter.

7. A system comprising:
a wireless charging circuit coupled to a wireless charging antenna and configured to transmit a wireless power signal from the wireless charging antenna to a wireless power receiving antenna based on a charging waveform from the wireless charging circuit;
a circuit for detecting a transition between an internal power source for the wireless charging circuit and an external power source for the wireless charging circuit; and
a controller configured to respond to the transition between the internal power source and the external power source by switching between a first waveform for driving the wireless charging circuit and a second waveform for driving the wireless charging circuit.

8. The system of claim 7, wherein the controller switches to the first waveform in response to the wireless charging circuit transitioning to the internal power source and switches to the second waveform in response to the wireless charging circuit transitioning to the external power source.

9. The system of claim 8, wherein the first waveform for the internal power source includes a square wave.

10. The system of claim 8, wherein the second waveform for the external power source includes a sine wave.

11. The system of claim 8, wherein the second waveform has fewer harmonics than the first waveform.

12. The system of claim 8, wherein the second waveform has fewer high frequency components than the first waveform.

13. The system of claim 8, wherein the second waveform has greater spectral concentration than the first waveform.

14. The system of claim 8, wherein the first waveform has greater spectral dispersion than the second waveform.

15. The system of claim 7, further comprising a connector for removably and replaceably coupling the wireless charging circuit to the external power source.

16. The system of claim 15, wherein detecting the transition includes detecting when the connector is connected to or disconnected from the external power source.

17. A method comprising:
driving a wireless charging circuit with a charging waveform to wirelessly transmit power;
detecting a transition between an internal power source for operating the wireless charging circuit and an external power source for operating the wireless charging circuit, wherein the external power source provides power to the wireless charging circuit through a cable; and
in response to detecting the transition, switching between a first waveform for driving the wireless charging circuit and a second waveform for driving the wireless charging circuit, wherein the first waveform has a greater spectral dispersion than the second waveform.

18. The method of claim 17, wherein the second waveform has fewer harmonics than the first waveform.

19. The method of claim 17, wherein the second waveform has fewer high frequency components than the first waveform.

20. A computer program product comprising computer-executable code stored in a non-transitory computer-readable medium that, when executing on one or more computing devices, causes the one or more computing devices to perform the steps of:
driving a wireless charging circuit with a charging waveform to wirelessly transmit power;
detecting a transition between an internal power source for operating the wireless charging circuit and an external power source for operating the wireless charging circuit, wherein the external power source provides power to the wireless charging circuit through a cable; and
in response to detecting the transition, switching between a first waveform for driving the wireless charging circuit and a second waveform for driving the wireless charging circuit, wherein the first waveform has a greater spectral dispersion than the second waveform.
